(19) United States
(12) Patent Application Publication
(10) Pub. No.: US 2022/0364044 A1
(43) Pub. Date: Nov. 17, 2022

(54) METHODS AND COMPOSITIONS FOR ANAEROBIC BACTERIAL FERMENTATION

(71) Applicant: Evelo Biosciences, Inc., Cambridge, MA (US)

(72) Inventors: Mehmedalija Jahic, Cambridge, MA (US); Dacvid Emerson, Cambridge, MA (US); Collin McKenna, Cambridge, MA (US); Raashed Raziuddin, Somerville, MA (US)

(21) Appl. No.: 17/613,341

(22) PCT Filed: May 21, 2020
(86) PCT No.: PCT/US20/33927

§ 371 Date: Nov. 22, 2021

Related U.S. Application Data
(60) Provisional application No. 62/952,798, filed on Dec. 23, 2019.
(60) Provisional application No. 62/850,726, filed on May 21, 2019.

(51) Int. Cl. C12N 1/20; C12M 1/34; C12N 1/38

(52) U.S. Cl. CPC C12N 1/20; C12M 41/34; C12N 1/38

(57) ABSTRACT

Provided herein are methods and compositions related to fermentation of anaerobic bacteria.

### REFERENCE TO RELATED APPLICATIONS

This application claims the benefit of U.S. Provisional Patent Application No. 62/850,726, filed May 21, 2019, and U.S. Provisional Patent Application No. 62/952,798, filed Dec. 23, 2019, the contents of each of which are hereby incorporated by reference in their entirety.

### BACKGROUND

Anaerobic bacteria are bacteria that that grow poorly (or do not grow) in the presence of oxygen. In humans, many types of anaerobic bacteria are found in the gastrointestinal tract. As microbial culturing methods typically occur in atmospheric air (an aerobic environment), the culturing of anaerobic bacteria can be challenging and often requires specialized equipment and techniques. For example, anaerobic bacteria can be cultured in an anaerobic glovebox or other specially sealed container filled with nitrogen. However, currently available techniques are not amenable to the large-scale cultures necessary for the commercial production of therapeutic microbes. Therefore, alternative methods for anaerobic bacterial fermentation would be useful for growing anaerobic bacteria, particularly in large scales.

### SUMMARY

Anaerobic bacteria benefit from the presence of carbon dioxide (CO₂) at the start of culturing in the lag phase, but some strains of anaerobic bacteria do not need CO₂ to maintain robust growth through log phase. Certain anaerobic strains, e.g., strains described herein, grow better when CO₂ is provided throughout growth (e.g., as compared to the rate of growth when CO₂ is not provided in log phase). For example, certain such bacteria consume CO₂ throughout the fermentation process.

In certain aspects, the culture methods described herein allow for better growth of an anaerobic bacteria strain, e.g., a strain described herein, as compared to conventional methods. For example, in some embodiments, the methods described herein allow growth of the bacteria to an OD of over 4, e.g., over 10, or over 20. For example, in some embodiments, sparging CO₂ at about 25% (e.g., and about 75% N₂) rather than at about 5% (e.g., and about 95% N₂) into a bioreactor allows about a 5-fold increase in biomass yield. The CO₂ can be introduced in a gas mixture; the gas mixture can also include N₂.

A key feature of certain embodiments of the methods described herein is that they are particularly applicable to large scale production, e.g., in a bioreactor, e.g., in vessels over 1 L in volume. As culture volumes increase, simply providing CO₂ into the headspace of a vessel may not suffice to provide sufficient CO₂ throughout the culture to achieve optimal growth. In some embodiments, by providing CO₂ throughout the culture (e.g., beyond providing CO₂ in the headspace), bacterial growth is improved. In some embodiments, CO₂ can be provided throughout the culture, e.g., by sparging/bubbling CO₂ into the culture; by injecting boluses of CO₂ into the culture at intervals (e.g., at 30-minute or one-hour intervals); and/or by adding a carbonate or bicarbonate salt into the culture. Carbonate salts that can be used in embodiments provided herein include, for example, sodium carbonate, potassium carbonate, barium carbonate, carbonic acid, magnesite (magnesium carbonate), sodium percarbonate (adduct with hydrogen peroxide) and calcium carbonate. Bicarbonate salts that can be used in embodiments provided herein include, for example, sodium bicarbonate, potassium bicarbonate, cesium bicarbonate, magnesium bicarbonate, and ammonium bicarbonate. The carbonate or bicarbonate salt can be used at a concentration of, e.g., 0.5 g/L to 10 g/L, e.g., 0.5 to 1 g/L, 1 to 5 g/L, 2 to 8 g/L, about 0.5 g/L, about 1 g/L, about 5 g/L, about 10 g/L. The carbonate or bicarbonate salt can be used in certain embodiments as an alternative or additional source of CO₂ (e.g., by adding the salt, a lower percentage of CO₂ can be used yet still achieve the same growth benefits as when a higher percentage of CO₂ is used). For example, in some embodiments, bacteria can be grown in a bioreactor into which about 25% CO₂ (e.g., and about 75% N₂) is sparged into the culture; similar yields can be obtained, e.g., growing the bacteria in a bioreactor into which about 5% CO₂ (e.g., and about 95% N₂) is sparged with the addition of sodium bicarbonate (e.g., 0.5-1 g/L).

In certain aspects, provided herein are methods of growing anaerobic bacteria comprising culturing the anaerobic bacteria in a bioreactor comprising carbonate salt. In some embodiments, is sodium carbonate, potassium carbonate, barium carbonate, carbonic acid, magnesite (magnesium carbonate), sodium percarbonate (adduct with hydrogen peroxide), or calcium carbonate. In some embodiments, the carbonate is at a concentration of 0.5 g/L to 10 g/L. In some embodiments, the carbonate salt is at a concentration of 0.5 to 1 g/L, 1 to 5 g/L, or 2 to 8 g/L. In some embodiments, the carbonate salt is at a concentration of about 0.5 g/L, about 1 g/L, about 5 g/L, or about 10 g/L.

In certain aspects, provided herein are methods of growing anaerobic bacteria comprising culturing the anaerobic bacteria in a bioreactor comprising bicarbonate salt. In some embodiments, the bicarbonate salt is sodium bicarbonate, potassium bicarbonate, cesium bicarbonate, magnesium bicarbonate, or ammonium bicarbonate. In some embodiments, the bicarbonate salt is at a concentration of 0.5 g/L to 10 g/L. In some embodiments, the bicarbonate salt is at a concentration of 0.5 to 1 g/L, 1 to 5 g/L, or 2 to 8 g/L. In some embodiments, the bicarbonate salt is at a concentration of about 0.5 g/L, about 1 g/L, about 5 g/L, or about 10 g/L.

In certain aspects, provided herein are improved compositions and methods for culturing anaerobic bacteria. For example, in some embodiments provided herein are methods of culturing anaerobic bacteria under anaerobic conditions comprising a greater level of CO₂ compared to conventional anaerobic culture conditions (e.g., at a level of greater than 1% CO₂, e.g., at a level of greater than 5% CO₂, such as at a level of about 25% CO₂). In certain embodiments, provided herein are bioreactors comprising anaerobic bacteria being cultured under conditions comprising a greater level of CO₂ compared to conventional anaerobic culture conditions (e.g., at a level of greater than 1% CO₂, such as at a level of about 25% CO₂). In some embodiments, the methods and compositions provided herein result in increased bacterial yield compared to conventional culture conditions.

In certain aspects, provided herein are methods of growing anaerobic bacteria comprising culturing the anaerobic bacteria in a bioreactor under an anaerobic atmosphere comprising CO₂. In some embodiments, the anaerobic atmosphere comprises greater than 1% CO₂. In some embodiments, the anaerobic atmosphere comprises greater than 5% CO₂. In some embodiments, the anaerobic atmosphere comprises at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% CO₂. In some embodiments, the anaerobic atmosphere comprises at least 8% CO₂. In some embodiments, the anaerobic atmosphere comprises at least 20% CO₂. In some embodiments, the anaerobic atmosphere comprises from 8% to 40% CO₂. In some embodiments, the anaerobic atmosphere comprises at least 10% CO₂. In some embodiments, the anaerobic atmosphere comprises at least 20% CO₂. In some embodiments, the anaerobic atmosphere comprises from 10% to 40% CO₂. In some embodiments, the anaerobic atmosphere comprises from 20% to 30% CO₂. In some embodiments, the anaerobic atmosphere comprises about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% CO₂. In some embodiments, the anaerobic atmosphere comprises about 25% CO₂.

In certain aspects, provided herein are methods of growing anaerobic bacteria comprising culturing the anaerobic bacteria in a bioreactor into which CO₂ in a gas mixture is introduced into the culture. In some embodiments, the gas mixture comprises greater than 1% CO₂. In some embodiments, the gas mixture comprises greater than 5% CO₂. In some embodiments, the gas mixture comprises at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% CO₂. In some embodiments, the gas mixture comprises at least 8% CO₂. In some embodiments, the gas mixture comprises at least 20% CO₂. In some embodiments, the gas mixture comprises from 8% to 40% CO₂. In some embodiments, the gas mixture comprises at least 10% CO₂. In some embodiments, the gas mixture comprises at least 20% CO₂. In some embodiments, the gas mixture comprises from 10% to 40% CO₂. In some embodiments, the gas mixture comprises from 20% to 30% CO₂. In some embodiments, the gas mixture comprises about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 50%, about 60%, about 70%, about 80%, about 90%, or about 100%, CO₂. In some embodiments, the gas mixture comprises about 25% CO₂.

In certain aspects, provided herein are methods of culturing anaerobic bacteria under anaerobic conditions comprising a lower level of N₂ compared to conventional anaerobic culture conditions (e.g., at a level of less than 95% N₂, e.g., at a level of less than 90% N₂, such as at a level of about 75% N₂). In certain embodiments, provided herein are bioreactors comprising anaerobic bacteria being cultured under conditions comprising a lower level of N₂ compared to conventional anaerobic culture conditions (e.g., at a level of less than 95% N₂ such as at a level of about 75% N₂). In some embodiments, the methods and compositions provided herein result in increased bacterial yield compared to conventional culture conditions.

In certain aspects, provided herein are methods of culturing anaerobic bacteria under anaerobic conditions comprising introducing a gas mixture comprising a lower level of N₂ compared to conventional anaerobic culture conditions (e.g., a gas mixture of less than 95% N₂, e.g., of less than 90% N₂, of about 75% N₂). In certain embodiments, provided herein are bioreactors comprising anaerobic bacteria being cultured under conditions comprising introducing a gas mixture comprising a lower level of N₂ compared to conventional anaerobic culture conditions (e.g., a gas mixture of less than 95% N₂ such as of about 75% N₂). In some embodiments, the methods and compositions provided herein result in increased bacterial yield compared to conventional culture conditions. In some embodiments, the gas mixture comprises no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, or no more than 94% N₂. In some embodiments, the gas mixture comprises from 75% to 94% N₂. In some embodiments, the gas mixture comprises about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, or about 94% N₂.

In certain aspects, provided herein are methods of growing anaerobic bacteria comprising culturing the anaerobic bacteria in a bioreactor under an anaerobic atmosphere comprising N₂. In some embodiments, the anaerobic atmosphere comprises less than 95% N₂. In some embodiments, the anaerobic atmosphere comprises less than 90% N₂. In some embodiments, the anaerobic atmosphere comprises less than 95%, less than 92%, less than 90%, less than 87%, less than 85%, less than 82%, less than 80%, less than 77% N₂. In some embodiments, the anaerobic atmosphere comprises less than 85% N₂. In some embodiments, the anaerobic atmosphere comprises less than 80% N₂. In some embodiments, the anaerobic atmosphere comprises from 65% to 85% N₂. In some embodiments, the anaerobic atmosphere comprises from 70% to 80% N₂. In some embodiments, the anaerobic atmosphere comprises about 65%, about 66%, about 67%, about 68%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the anaerobic atmosphere comprises about 75% N₂.

In certain aspects, provided herein are methods of growing anaerobic bacteria comprising culturing the anaerobic bacteria in a bioreactor into which an anaerobic gas mixture comprising N₂ is introduced. In some embodiments, the gas mixture comprises less than 95% N₂. In some embodiments, the gas mixture comprises less than 90% N₂. In some embodiments, the gas mixture comprises less than 95%, less than 92%, less than 90%, less than 87%, less than 85%, less than 82%, less than 80%, less than 77% N₂. In some embodiments, the gas mixture comprises less than 85% N₂. In some embodiments, the gas mixture comprises less than 80% N₂. In some embodiments, the gas mixture comprises from 65% to 85% N₂. In some embodiments, the gas mixture comprises from 70% to 80% N₂. In some embodiments, the gas mixture comprises about 65%, about 66%, about 67%, about 68%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the gas mixture comprises about 75% N₂.

In some embodiments, the anaerobic atmosphere and/or gas mixture consists essentially of CO₂ and N₂. In some embodiments, the anaerobic atmosphere and/or gas mixture comprises about 25% CO₂ and about 75% N₂. In some embodiments, the anaerobic atmosphere and/or gas mixture comprises about 20% CO₂ and about 80% N₂. In some embodiments, the anaerobic atmosphere and/or gas mixture comprises about 30% CO₂ and about 70% N₂.

In certain aspects, provided herein are methods of culturing anaerobic bacteria, the method comprising the steps of a) purging a bioreactor with an anaerobic gas mixture comprising greater than 1% CO₂; and b) culturing the anaerobic bacteria in the bioreactor purged in step a). In certain embodiments, the anaerobic gas mixture is added to the bioreactor during step b). In some embodiments, the anaerobic gas mixture comprises at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% CO₂. In some embodiments, the anaerobic gas mixture comprises greater than 5% CO₂. In some embodiments, the anaerobic gas mixture comprises at least 8% CO₂. In some embodiments, the anaerobic gas mixture comprises at least 20% CO₂. In some embodiments, the anaerobic gas mixture comprises from 8% to 40% CO₂. In some embodiments, the anaerobic gas mixture comprises from 20% to 30% CO₂. In some embodiments, the anaerobic gas mixture comprises about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 50%, about 60%, about 70%, about 80%, about 90%, or about 100%, CO₂. In some embodiments, the anaerobic gas mixture comprises about 25% CO₂. In some embodiments, the anaerobic gas mixture comprises about 100% CO₂.

In certain aspects, provided herein are methods of culturing anaerobic bacteria, the method comprising the steps of a) purging a bioreactor with an anaerobic gas mixture comprising less than 95% N₂; and b) culturing the anaerobic bacteria in the bioreactor purged in step a). In certain embodiments, the anaerobic gas mixture is added to the bioreactor during step b). In some embodiments, the anaerobic gas mixture comprises less than 95%, less than 92%, less than 90%, less than 87%, less than 85%, less than 82%, less than 80%, less than 77% N₂. In some embodiments, the anaerobic gas mixture comprises less than 85% N₂. In some embodiments, the anaerobic gas mixture comprises less than 80% N₂. In some embodiments, the anaerobic gas mixture comprises from 65% to 85% N₂. In some embodiments, the anaerobic gas mixture comprises from 70% to 80% N₂. In some embodiments, the anaerobic gas mixture comprises about 65%, about 66%, about 67%, about 28%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the anaerobic gas mixture comprises about 75% N₂.

In some embodiments, the anaerobic gas mixture consists essentially of CO₂ and N₂. In some embodiments, the anaerobic gas mixture comprises about 25% CO₂ and about 75% N₂. In some embodiments, the anaerobic atmosphere comprises about 20% CO₂ and about 80% N₂. In some embodiments, the anaerobic atmosphere comprises about 30% CO₂ and about 70% N₂.

In some embodiments, the methods provided herein further comprises the step of inoculating a growth media with the anaerobic bacteria, wherein the bacteria are cultured in the growth media according to the methods provided herein. In some embodiments, the volume of the inoculated anaerobic bacteria is between 0.01% and 10% v/v of the growth media (e.g., about 0.1% v/v of the growth media, about 0.5% v/v of the growth media, about 1% v/v of the growth media, about 5% v/v of the growth media).

In some embodiments, the growth media is at least about 1 L in volume, at least about 5 L in volume, at least about 10 L in volume, at least about 15 L in volume, at least about 20 L in volume, at least about 30 L in volume, at least about 40 L in volume, at least about 50 L in volume, at least about 100 L in volume, at least about 200 L in volume, at least about 250 L in volume, at least about 500 L in volume, at least about 750 L in volume, at least about 1000 L in volume, at least about 1500 L in volume, at least about 2000 L in volume, at least about 2500 L in volume, at least about 3000 L in volume, at least about 3500 L in volume, at least about 4000 L in volume, at least about 5000 L in volume, at least about 7500 L in volume, at least about 10,000 L in volume, at least about 15,000 L in volume, at least about 20,000 L in volume, at least about 50,000 L in volume, at least about 100,000 L in volume, at least about 150,000 L in volume, at least about 200,000 L in volume, at least about 250,000 L in volume, at least about 300,000 L in volume, at least about 350,000 L in volume, at least about 400,000 L in volume, or at least about 500,000 L in volume.

In some embodiments, the anaerobic bacteria is cultured for at least 5 hours (e.g., at least 10 hours). In some embodiments, the anaerobic bacteria is cultured for 10-24 hours. In some embodiments, the anaerobic bacteria is cultured for 14 to 16 hours. In some embodiments, the method further comprises the step of inoculating about 5% v/v of the cultured bacteria in a growth media. In some embodiments, the growth media is about 20 L in volume. In some embodiments, the anaerobic bacteria is cultured for 10-24 hours. In some embodiments, the anaerobic bacteria is cultured for 12-14 hours. In some embodiments, the anaerobic bacteria is cultured at least until a stationary phase is reached.

In some embodiments, the anaerobic bacteria is cultured at a temperature of 35° C. to 42° C. In some embodiments, the anaerobic bacteria is cultured at a temperature of 35° C. to 39° C. In some embodiments, the anaerobic bacteria is cultured at a temperature of about 37° C. In some embodiments, the anaerobic bacteria is cultured at a pH of 5.5 to 7.5. In some embodiments, the anaerobic bacteria is cultured at a pH of about 6.5.

In some embodiments, the anaerobic bacteria is cultured in a bioreactor. In some embodiments, culturing the anaerobic bacteria comprises agitating the culture at a RPM of 50 to 1000. In some embodiments, culturing the anaerobic bacteria comprises agitating the culture at a RPM of 100 to 700. In some embodiments, culturing the anaerobic bacteria comprises agitating the culture at a RPM of 50 to 300. In some embodiments, the anaerobic bacteria is agitated at a RPM of about 150.

In some embodiments, an anaerobic gas mixture is continuously added to the bioreactor during culturing. In some embodiments, the continuously added anaerobic gas mixture is added at a rate of 0.01 to 1 vvm. In some embodiments, the continuously added anaerobic gas mixture is added at a rate of 0.01 to 0.1 vvm. In some embodiments, the continuously added anaerobic gas mixture is added at a rate of 0.02 vvm. In some embodiments, CO₂ is continuously added during culturing. In some embodiments, CO₂ is added at a rate of 0.002 vvm to 0.1 vvm. In some embodiments, CO₂ is added at a rate of about 0.002 vvm. In some embodiments, CO₂ is added at a rate of about 0.02 vvm. In some embodiments, the continuously added anaerobic gas mixture comprises at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% CO₂. In some embodiments, the anaerobic gas mixture comprises greater than 5% CO₂. In some embodiments, the continuously added anaerobic gas mixture comprises at least 8% CO₂. In some embodiments, the continuously added anaerobic gas mixture comprises at least 20% CO₂. In some embodiments, the anaerobic atmosphere comprises from 8% to 40% CO₂. In some embodiments, the continuously added anaerobic gas mixture comprises at least 10% CO₂. In some embodiments, the continuously added anaerobic gas mixture comprises at least 20% CO₂. In some embodiments, the anaerobic atmosphere comprises from 10% to 40% CO₂. In some embodiments, the continuously added anaerobic gas mixture comprises from 20% to 30% CO₂. In some embodiments, the continuously added anaerobic gas mixture comprises about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 50%, about 60%, about 70%, about 80%, about 90%, or about 100% CO₂. In some embodiments, the continuously added anaerobic gas mixture comprises about 25% CO₂.

In some embodiments, an anaerobic gas mixture is continuously added to the bioreactor during culturing. In some embodiments, the continuously added anaerobic gas mixture is added at a rate of 0.01 to 0.1 vvm. In some embodiments the continuously added anaerobic gas mixture is added at a rate of about 0.02 vvm. In some embodiments, CO₂ is continuously added during culturing. In some embodiments, CO₂ is added at a rate of 0.002 vvm to 0.1 vvm. In some embodiments, CO₂ is added at a rate of about 0.002 vvm. In some embodiments, CO₂ is added at a rate of about 0.02 vvm. In some embodiments, CO₂ is added at a rate of about 0.007 vvm. In some embodiments, the continuously added anaerobic gas mixture comprises less than 95%, less than 92%, less than 90%, less than 87%, less than 85%, less than 82%, less than 80%, less than 77% N₂. In some embodiments, the anaerobic gas mixture comprises less than 85% N₂. In some embodiments, the continuously added anaerobic gas mixture comprises less than 80% N₂. In some embodiments, the anaerobic atmosphere comprises from 65% to 85% N₂. In some embodiments, the continuously added anaerobic gas mixture comprises from 70% to 80% N₂. In some embodiments, the continuously added anaerobic gas mixture comprises about 65%, about 66%, about 67%, about 28%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the continuously added anaerobic gas mixture comprises about 75% N₂.

In some embodiments, the anaerobic atmosphere consists essentially of CO₂ and N₂. In some embodiments, the continuously added anaerobic gas mixture comprises about 25% CO₂ and about 75% N₂. In some embodiments, the anaerobic atmosphere comprises about 20% CO₂ and about 80% N₂. In some embodiments, the anaerobic atmosphere comprises about 30% CO₂ and about 70% N₂.

In certain embodiments of the methods provided herein, herein the anaerobic bacteria are cultured in a pressurized bioreactor. In some embodiments, the bioreactor is pressurized at least at 100,000 Pascal. In some embodiments, the bioreactor is pressurized at least at 100,000 Pascal, 125,000 Pascal, 150,000 Pascal, 175,000 Pascal, 200,000 Pascal, or 225,000 Pascal. In some embodiments, the bioreactor is pressurized at most at 2,225,000 Pascal. In some embodiments, the bioreactor is pressurized at most at 2,000,000 Pascal, 2,025,000 Pascal, 2,050,000 Pascal, 2,075,000 Pascal, 2,100,000 Pascal, 2,150,000 Pascal, 2,200,000 Pascal, or 2,225,000 Pascal. In some embodiments, the bioreactor is pressurized from about 100,000 Pascal to about 2,100,000 Pascal. In some embodiments, the bioreactor is pressurized from about 101,325 Pascal to about 2,026,500 Pascal. Generally, but in no way wishing to be bound by theory, operating at increased pressures allows a significant increase in the rate of CO₂ transfer from the gas phase to the liquid phase.

In certain embodiments, the methods provided herein comprise introducing a gas to the bioreactor with a diffusion sparger. In some embodiments, the gas is introduced with sintered or porous spargers. In other embodiments, the gas is introduced with perforated plates or other apparatus to introduce microbubbles. Generally, but in no way wishing to be bound by theory, the introduction of smaller and more diffuse bubbles allows a significant increase in the rate of CO₂ transfer from the gas phase to the liquid phase.

In some embodiments the anaerobic bacteria is cultured in growth media. In some embodiments, the growth media comprises yeast extract, soy peptone A2SC 19649, Soy peptone E110 19885, dipotassium phosphate, monopotassium phosphate, L-cysteine-HCl, ammonium chloride, maltodextrin (e.g., glucidex, e.g., glucidex 21 D), glucose, and/or hemoglobin. In some embodiments, the growth media comprises 5 g/L to 15 g/L yeast extract 19512. In some embodiments, the growth media comprises about 10 g/L yeast extract 19512. In some embodiments, the growth media comprises 10 g/L to 15 g/L soy peptone A2SC 19649. In some embodiments, the growth media comprises about 12.5 g/L soy peptone A2SC 19649. In some embodiments, the growth media comprises 10 g/L to 15 g/L Soy peptone E110 19885. In some embodiments, the growth media comprises about 12.5 g/L Soy peptone E110 19885. In some embodiments, the growth media comprises 1 g/L to 2 g/L dipotassium phosphate. In some embodiments, the growth media comprises about 1.59 g/L Dipotassium phosphate. In some embodiments, the growth media comprises 0.5 g/L to 1.5 g/L monopotassium phosphate. In some embodiments, the growth media comprises about 0.91 g/L monopotassium phosphate. In some embodiments, the growth media comprises 0.1 g/L to 1.0 g/L L-cysteine-HCl. In some embodiments, the growth media comprises about 0.5 g/L L-cysteine-HCl. In some embodiments, the growth media comprises 0.1 g/L to 1.0 g/L ammonium chloride. In some embodiments, the growth media comprises about 0.5 g/L ammonium chloride. In some embodiments, the growth media comprises 20 g/L to 30 g/L maltodextrin (e.g., glucidex, e.g., glucidex 21 D). In some embodiments, the growth media comprises about 25 g/L maltodextrin (e.g., glucidex, e.g., glucidex 21 D). In some embodiments, the growth media comprises 5 g/L to 15 g/L glucose. In some embodiments, the growth media comprises about 10 g/L glucose. In some embodiments, the growth media comprises 0.01 g/L to 0.05 g/L hemoglobin. In some embodiments, the growth media comprises about 0.02 g/L hemoglobin.

In some embodiments the anaerobic bacteria is cultured in growth media. In some embodiments, the growth media comprises yeast extract, soy peptone A2SC 19649, Soy peptone E110 19885, dipotassium phosphate, monopotassium phosphate, L-cysteine-HCl, ammonium chloride, glucose, and/or hemoglobin. In some embodiments, the growth media comprises 5 g/L to 15 g/L yeast extract 19512. In some embodiments, the growth media comprises about 10 g/L yeast extract 19512. In some embodiments, the growth media comprises 10 g/L to 15 g/L soy peptone A2SC 19649. In some embodiments, the growth media comprises about 12.5 g/L soy peptone A2SC 19649. In some embodiments, the growth media comprises 10 g/L to 15 g/L Soy peptone E110 19885. In some embodiments, the growth media comprises about 12.5 g/L Soy peptone E110 19885. In some embodiments, the growth media comprises 1 g/L to 2 g/L dipotassium phosphate. In some embodiments, the growth media comprises about 1.59 g/L Dipotassium phosphate. In some embodiments, the growth media comprises 0.5 g/L to 1.5 g/L monopotassium phosphate. In some embodiments, the growth media comprises about 0.91 g/L monopotassium phosphate. In some embodiments, the growth media comprises 0.1 g/L to 1.0 g/L L-cysteine-HCl. In some embodiments, the growth media comprises about 0.5 g/L L-cysteine-HCl. In some embodiments, the growth media comprises 0.1 g/L to 1.0 g/L ammonium chloride. In some embodiments, the growth media comprises about 0.5 g/L ammonium chloride. In some embodiments, the growth media comprises 5 g/L to 15 g/L glucose. In some embodiments, the growth media comprises about 10 g/L glucose. In some embodiments, the growth media comprises 0.01 g/L to 0.05 g/L hemoglobin. In some embodiments, the growth media comprises about 0.02 g/L hemoglobin.

In some embodiments the anaerobic bacteria is cultured in growth media. In some embodiments, the growth media comprises yeast extract, soy peptone A2SC 19649, Soy peptone E110 19885, dipotassium phosphate, monopotassium phosphate, L-cysteine-HCl, ammonium chloride, maltodextrin (e.g., glucidex, e.g., glucidex 21 D), and/or hemoglobin. In some embodiments, the growth media comprises 5 g/L to 15 g/L yeast extract 19512. In some embodiments, the growth media comprises about 10 g/L yeast extract 19512. In some embodiments, the growth media comprises 10 g/L to 15 g/L soy peptone A2SC 19649. In some embodiments, the growth media comprises about 12.5 g/L soy peptone A2SC 19649. In some embodiments, the growth media comprises 10 g/L to 15 g/L Soy peptone E110 19885. In some embodiments, the growth media comprises about 12.5 g/L Soy peptone E110 19885. In some embodiments, the growth media comprises 1 g/L to 2 g/L dipotassium phosphate. In some embodiments, the growth media comprises about 1.59 g/L Dipotassium phosphate. In some embodiments, the growth media comprises 0.5 g/L to 1.5 g/L monopotassium phosphate. In some embodiments, the growth media comprises about 0.91 g/L monopotassium phosphate. In some embodiments, the growth media comprises 0.1 g/L to 1.0 g/L L-cysteine-HCl. In some embodiments, the growth media comprises about 0.5 g/L L-cysteine-HCl. In some embodiments, the growth media comprises 0.1 g/L to 1.0 g/L ammonium chloride. In some embodiments, the growth media comprises about 0.5 g/L ammonium chloride. In some embodiments, the growth media comprises 20 g/L to 30 g/L maltodextrin (e.g., glucidex, e.g., glucidex 21 D). In some embodiments, the growth media comprises about 25 g/L maltodextrin (e.g., glucidex, e.g., glucidex 21 D). In some embodiments, the growth media comprises 0.01 g/L to 0.05 g/L hemoglobin. In some embodiments, the growth media comprises about 0.02 g/L hemoglobin.

In some embodiments, the method further comprises the step of harvesting the cultured bacteria (e.g., when a stationary phase is reached). In some embodiments, the method further comprises the step of centrifuging the cultured bacteria after harvesting (e.g., to produce a cell paste). In some embodiments, the method further comprises diluting the cell paste with a stabilizer solution to produce a cell slurry. In some embodiments, the method further comprises the step of lyophilizing the cell slurry to produce a powder. In some embodiments, the method further comprises irradiating the powder with gamma radiation.

In certain aspects, provided herein are bioreactors comprising anaerobic bacteria under an anaerobic atmosphere comprising at least about 1% CO₂. In some embodiments, the anaerobic atmosphere comprises at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% CO₂. In some embodiments, the anaerobic gas mixture comprises greater than 5% CO₂. In some embodiments, the anaerobic atmosphere comprises at least 8% CO₂. In some embodiments, the anaerobic atmosphere comprises at least 10% CO₂. In some embodiments, the anaerobic atmosphere comprises at least 20% CO₂. In some embodiments, the anaerobic atmosphere comprises from 8% to 40% CO₂. In some embodiments, the anaerobic atmosphere comprises from 10% to 40% CO₂. In some embodiments, the anaerobic atmosphere comprises from 20% to 30% CO₂. In some embodiments, the anaerobic atmosphere comprises about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 50%, about 60%, about 70%, about 80%, about 90%, or about 100%, CO₂. In some embodiments, the anaerobic atmosphere comprises about 25% CO₂.

In certain aspects, provided herein are bioreactors comprising anaerobic bacteria under an anaerobic atmosphere comprising less than 95% N₂. In some embodiments, the anaerobic atmosphere comprises less than 90% N₂. In some embodiments, the anaerobic atmosphere comprises less than 95%, less than 92%, less than 90%, less than 87%, less than 85%, less than 82%, less than 80%, less than 77% N₂. In some embodiments, the anaerobic gas mixture comprises less than 85% N₂. In some embodiments, the anaerobic atmosphere comprises less than 80% N₂. In some embodiments, the anaerobic atmosphere comprises from 65% to 85% N₂. In some embodiments, the anaerobic atmosphere comprises from 70% to 80% N₂. In some embodiments, the anaerobic atmosphere comprises about 65%, about 66%, about 67%, about 28%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the anaerobic atmosphere comprises about 75% N₂.

In some embodiments, the anaerobic atmosphere consists essentially of CO₂ and N₂. In some embodiments, the anaerobic atmosphere comprises about 25% CO₂ and about 75% N₂. In some embodiments, the anaerobic atmosphere comprises about 20% CO₂ and about 80% N₂. In some embodiments, the anaerobic atmosphere comprises about 30% CO₂ and about 70% N₂.

In some embodiments, the bioreactor is at least about 1 L in volume, at least about 5 L in volume, at least about 10 L in volume, at least about 15 L in volume, at least about 20 L in volume, at least about 30 L in volume, at least about 40 L in volume, at least about 50 L in volume, at least about 100 L in volume, at least about 200 L in volume, at least about 250 L in volume, at least about 500 L in volume, at least about 750 L in volume, at least about 1000 L in volume, at least about 1500 L in volume, at least about 2000 L in volume, at least about 2500 L in volume, at least about 3000 L in volume, at least about 3500 L in volume, at least about 4000 L in volume, at least about 5000 L in volume, at least about 7500 L in volume, at least about 10,000 L in volume, at least about 15,000 L in volume, at least about 20,000 L in volume, at least about 30,000 L in volume, at least about 50,000 L in volume, at least about 100,000 L in volume, at least about 150,000 L in volume, at least about 200,000 L in volume, at least about 250,000 L in volume, at least about 300,000 L in volume, at least about 350,000 L in volume, at least about 400,000 L in volume, at least about 450,000 L in volume or at least about 500,000 L in volume. In some embodiments, the bioreactor is an about 20 L bioreactor, an about 3500 L bioreactor, an about 20,000 L bioreactor, an about 50,000 L bioreactor, an about 100,000 L bioreactor, an about 200,000 L bioreactor, an about 300,000 L bioreactor, an about 400,000 L bioreactor or an about 500,000 L bioreactor. In some embodiments, the bioreactor is an about 20 L bioreactor, an about 3500 L bioreactor, an about 20,000 L bioreactor, or an about 400,000 L bioreactor.

At all scales, mass transfer of CO₂ can be important and is determined by a variety of factors. For example, mass transfer of CO₂ can be modulated by other factors including, but not limited to, increasing gas flow, increasing the concentration of CO₂ in the gas, increasing media agitation, agitator geometry, reactor geometry, and using a scintillator or other device to create smaller CO₂ gas bubbles. Alternatively, addition of bicarbonate or other sources of CO₂ can be implemented prior to or during culture growth. In certain embodiments, a combination specific to the vessel hardware/configuration can be used to optimize growth.

In some embodiments, the bioreactor further comprises a growth media. In some embodiments, the growth media comprises yeast extract, soy peptone A2SC 19649, Soy peptone E110 19885, dipotassium phosphate, monopotassium phosphate, L-cysteine-HCl, ammonium chloride, maltodextrin (e.g., glucidex, e.g., glucidex 21 D), glucose, and hemoglobin. In some embodiments, the growth media comprises 5 g/L to 15 g/L yeast extract 19512. In some embodiments, the growth media comprises about 10 g/L yeast extract 19512. In some embodiments, the growth media comprises 10 g/L to 15 g/L soy peptone A2SC 19649. In some embodiments, the growth media comprises about 12.5 g/L soy peptone A2SC 19649. In some embodiments, the growth media comprises 10 g/L to 15 g/L Soy peptone E110 19885. In some embodiments, the growth media comprises about 12.5 g/L Soy peptone E110 19885. In some embodiments, the growth media comprises 1 g/L to 2 g/L dipotassium phosphate. In some embodiments, the growth media comprises about 1.59 g/L Dipotassium phosphate. In some embodiments, the growth media comprises 0.5 g/L to 1.5 g/L monopotassium phosphate. In some embodiments, the growth media comprises about 0.91 g/L monopotassium phosphate. In some embodiments, the growth media comprises 0.1 g/L to 1.0 g/L L-cysteine-HCl. In some embodiments, the growth media comprises about 0.5 g/L L-cysteine-HCl. In some embodiments, the growth media comprises 0.1 g/L to 1.0 g/L ammonium chloride. In some embodiments, the growth media comprises about 0.5 g/L ammonium chloride. In some embodiments, the growth media comprises 20 g/L to 30 g/L maltodextrin (e.g., glucidex, e.g., glucidex 21 D). In some embodiments, the growth media comprises about 25 g/L maltodextrin (e.g., glucidex, e.g., glucidex 21 D). In some embodiments, the growth media comprises 5 g/L to 15 g/L glucose. In some embodiments, the growth media comprises about 10 g/L glucose. In some embodiments, the growth media comprises 0.01 g/L to 0.05 g/L hemoglobin. In some embodiments, the growth media comprises about 0.02 g/L hemoglobin.

In some embodiments the anaerobic bacteria is cultured in growth media. In some embodiments, the growth media comprises yeast extract, soy peptone A2SC 19649, Soy peptone E110 19885, dipotassium phosphate, monopotassium phosphate, L-cysteine-HCl, ammonium chloride, glucose, and/or hemoglobin. In some embodiments, the growth media comprises 5 g/L to 15 g/L yeast extract 19512. In some embodiments, the growth media comprises about 10 g/L yeast extract 19512. In some embodiments, the growth media comprises 10 g/L to 15 g/L soy peptone A2SC 19649. In some embodiments, the growth media comprises about 12.5 g/L soy peptone A2SC 19649. In some embodiments, the growth media comprises 10 g/L to 15 g/L Soy peptone E110 19885. In some embodiments, the growth media comprises about 12.5 g/L Soy peptone E110 19885. In some embodiments, the growth media comprises 1 g/L to 2 g/L dipotassium phosphate. In some embodiments, the growth media comprises about 1.59 g/L Dipotassium phosphate. In some embodiments, the growth media comprises 0.5 g/L to 1.5 g/L monopotassium phosphate. In some embodiments, the growth media comprises about 0.91 g/L monopotassium phosphate. In some embodiments, the growth media comprises 0.1 g/L to 1.0 g/L L-cysteine-HCl. In some embodiments, the growth media comprises about 0.5 g/L L-cysteine-HCl. In some embodiments, the growth media comprises 0.1 g/L to 1.0 g/L ammonium chloride. In some embodiments, the growth media comprises about 0.5 g/L ammonium chloride. In some embodiments, the growth media comprises 5 g/L to 15 g/L glucose. In some embodiments, the growth media comprises about 10 g/L glucose. In some embodiments, the growth media comprises 0.01 g/L to 0.05 g/L hemoglobin. In some embodiments, the growth media comprises about 0.02 g/L hemoglobin.

In some embodiments the anaerobic bacteria is cultured in growth media. In some embodiments, the growth media comprises yeast extract, soy peptone A2SC 19649, Soy peptone E110 19885, dipotassium phosphate, monopotassium phosphate, L-cysteine-HCl, ammonium chloride, maltodextrin (e.g., glucidex, e.g., glucidex 21 D), and/or hemoglobin. In some embodiments, the growth media comprises 5 g/L to 15 g/L yeast extract 19512. In some embodiments, the growth media comprises about 10 g/L yeast extract 19512. In some embodiments, the growth media comprises 10 g/L to 15 g/L soy peptone A2SC 19649. In some embodiments, the growth media comprises about 12.5 g/L soy peptone A2SC 19649. In some embodiments, the growth media comprises 10 g/L to 15 g/L Soy peptone E110 19885. In some embodiments, the growth media comprises about 12.5 g/L Soy peptone E110 19885. In some embodiments, the growth media comprises 1 g/L to 2 g/L dipotassium phosphate. In some embodiments, the growth media comprises about 1.59 g/L Dipotassium phosphate. In some embodiments, the growth media comprises 0.5 g/L to 1.5 g/L monopotassium phosphate. In some embodiments, the growth media comprises about 0.91 g/L monopotassium phosphate. In some embodiments, the growth media comprises 0.1 g/L to 1.0 g/L L-cysteine-HCl. In some embodiments, the growth media comprises about 0.5 g/L L-cysteine-HCl. In some embodiments, the growth media comprises 0.1 g/L to 1.0 g/L ammonium chloride. In some embodiments, the growth media comprises about 0.5 g/L ammonium chloride. In some embodiments, the growth media comprises 20 g/L to 30 g/L maltodextrin (e.g., glucidex, e.g., glucidex 21 D). In some embodiments, the growth media comprises about 25 g/L maltodextrin (e.g., glucidex, e.g., glucidex 21 D). In some embodiments, the growth media comprises 0.01 g/L to 0.05 g/L hemoglobin. In some embodiments, the growth media comprises about 0.02 g/L hemoglobin.

In some embodiments, the anaerobic bacteria are selected from bacteria of the genus Actinomyces, Bacteroides, Clostridium, Fusobacterium, Peptostreptococcus, Porphyromonas, Prevotella, Propionibacterium, or Veillonella. In some embodiments, the anaerobic bacteria are from the genus Prevotella. In some embodiments, the anaerobic bacteria are from a strain of Prevotella bacteria comprising one or more proteins listed in Table 1. In some embodiments, the anaerobic bacteria are from a strain of Prevotella substantially free of a protein listed in Table 2. In some embodiments, the anaerobic bacteria are from a strain of Prevotella bacteria comprising one or more of the proteins listed in Table 1 and that is free or substantially free of a protein listed in Table 2.

In some embodiments, the Prevotella bacteria are of the species Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

In some embodiments, the Prevotella is Prevotella Strain B 50329 (NRRL accession number B 50329). In some embodiments, the Prevotella strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

In some embodiments, the Prevotella bacteria is a strain of Prevotella bacteria comprising a protein listed in Table 1 and/or a gene encoding a protein listed in Table 1. In some embodiments, the Prevotella bacteria is a strain of Prevotella bacteria free or substantially free of a protein listed in Table 2 and/or a gene encoding a protein listed in Table 2.

In some aspects, provided herein is a stabilizer that stabilizes bacterial compositions and methods of making and using such a stabilizer. In some embodiments, the stabilizer comprises at least one of sucrose, dextran 40k, cysteine HCl, and water. In some embodiments, the stabilizer comprises sucrose (e.g., about 200 g/kg sucrose), dextran 40k (e.g., about 200 g/kg dextran 40k), cysteine HCl (about 4 g/kg cysteine HCl), and water (e.g., about 596 g/kg water). In some aspects, provided herein are bacterial compositions comprising a stabilizer provided herein and bacteria (e.g., a Prevotella strain disclosed herein), and methods of preparing the same. In some embodiments, the bacterial composition comprises sucrose, dextran 40k, and cysteine HCl. In some embodiments, the bacterial composition comprises 1.5% sucrose, 1.5% dextran 40k, and 0.03% cysteine HCl. In certain embodiments, the bacterial composition is prepared by combining and mixing bacteria with a certain percentage of the stabilizer in liquid suspension. In some embodiments, the percentage of the stabilizer solution used to mix with bacteria is about 10%. In some embodiments, the bacteria in the bacterial composition are anaerobic bacteria. In some embodiments, the anaerobic bacteria are Prevotella histicola. In some such embodiments, the anaerobic bacteria are Prevotella histicola Strain B 50329. In some embodiments, the bacterial composition is lyophilized to form a powder.

### BRIEF DESCRIPTION OF THE FIGURES

FIG. 1 is a schematic of an exemplary manufacturing process for anaerobic bacteria, including, e.g., Prevotella histicola.

FIG. 2 is a schematic of an exemplary manufacturing process described herein.

FIG. 3 is a plot showing that reduced rates of sparging (bubbling) of 95% N₂, 5% CO₂ gas (0.1 vvm vs. 0.02 vvm) results in decreased growth potential of Prevotella histicola Strain B 50329 anaerobic bacteria. (vvm stands for Volume of gas per Volume of vessel per Minute).

FIG. 4 is a plot showing that the presence of CO₂ is necessary for initiating Prevotella histicola Strain B 50329 growth, as well as the effect of various amounts of CO₂ (0%, 5%, 25%, 100%) on Prevotella histicola growth potential. (vvm stands for volume of gas per volume of vessel per minute).

FIG. 5 is a plot showing that Prevotella histicola Strain B 50329 consumes CO₂.

FIG. 6 is a plot showing that maltodextrin in combination with glucose can support growth of Prevotella histicola Strain B 50329 better than glucose alone.

### DETAILED DESCRIPTION

### Definitions

As used herein, “anaerobic conditions” are conditions with reduced levels of oxygen compared to normal atmospheric conditions. For example, in some embodiments anaerobic conditions are conditions wherein the oxygen levels are partial pressure of oxygen (pO₂) no more than 8%. In some instances, anaerobic conditions are conditions wherein the pO₂ is no more than 2%. In some instances, anaerobic conditions are conditions wherein the pO₂ is no more than 0.5%. In certain embodiments, anaerobic conditions may be achieved by purging a bioreactor and/or a culture flask with a gas other than oxygen such as, for example, nitrogen and/or carbon dioxide (CO₂).

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state.

As used herein, “engineered bacteria” are any bacteria that have been genetically altered from their natural state by human intervention and the progeny of any such bacteria. Engineered bacteria include, for example, the products of targeted genetic modification, the products of random mutagenesis screens and the products of directed evolution.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }9 fold, 10\hat{ }4 fold, 10\hat{ }5 fold, 10\hat{ }6 fold, and/or 10\hat{ }7 fold greater after treatment when compared to a pre-treatment state. Properties that may be increased include immune cells, bacterial cells, stromal cells, myeloid derived suppressor cells, fibroblasts, metabolites, and cytokines.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g. Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic Glade are provided herein.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

### Manufacturing Process

In certain aspects, provided herein are methods of culturing anaerobic bacteria comprising culturing the anaerobic bacteria in a bioreactor under an anaerobic atmosphere comprising CO₂, e.g., greater than 1% CO₂ (e.g., greater than 5% CO₂). In certain aspects, provided herein are methods of growing anaerobic bacteria comprising culturing the anaerobic bacteria in a bioreactor into which a gas mixture comprising CO₂ is introduced e.g., greater than 1% CO₂ (e.g., greater than 5% CO₂). In some embodiments, provided herein are methods culturing anaerobic bacteria, the method comprising the steps of a) purging a bioreactor with an anaerobic gas mixture comprising greater than 1% CO₂; and b) culturing the anaerobic bacteria in the bioreactor purged in step a) (e.g., while a gas mixture comprising greater than 1% CO₂ is introduced into the bioreactor).

In certain aspects, provided herein are methods of culturing anaerobic bacteria comprising culturing the anaerobic bacteria in a bioreactor under an anaerobic atmosphere comprising N₂, e.g., less than 95% N₂ (e.g., less than 90% N₂). In certain aspects, provided herein are methods of culturing anaerobic bacteria comprising culturing the anaerobic bacteria in a bioreactor into which a gas mixture comprising N₂ is introduced, e.g., less than 95% N₂ (e.g., less than 90% N₂). In some embodiments, provided herein are methods culturing anaerobic bacteria, the method comprising the steps of a) purging a bioreactor with an anaerobic gas mixture comprising less than 95% N₂; and b) culturing the anaerobic bacteria in the bioreactor purged in step a) (e.g., while a gas mixture comprising less than 95% N₂ is introduced into the bioreactor).

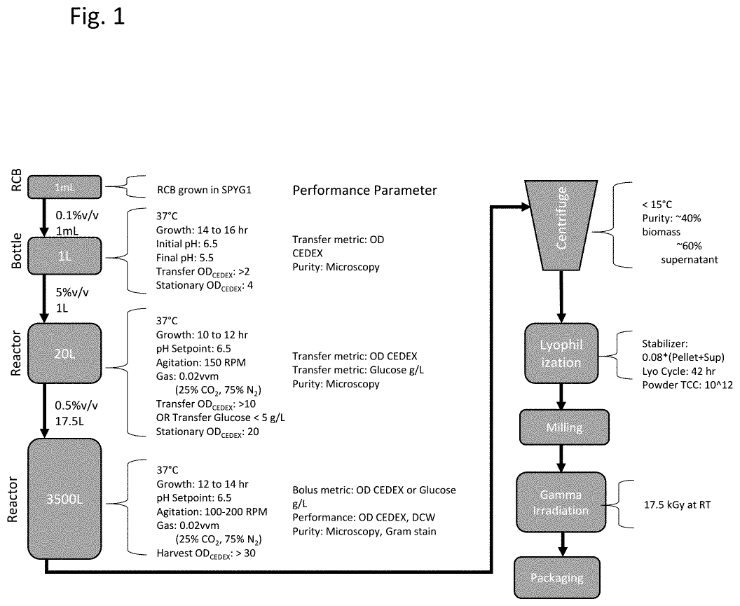

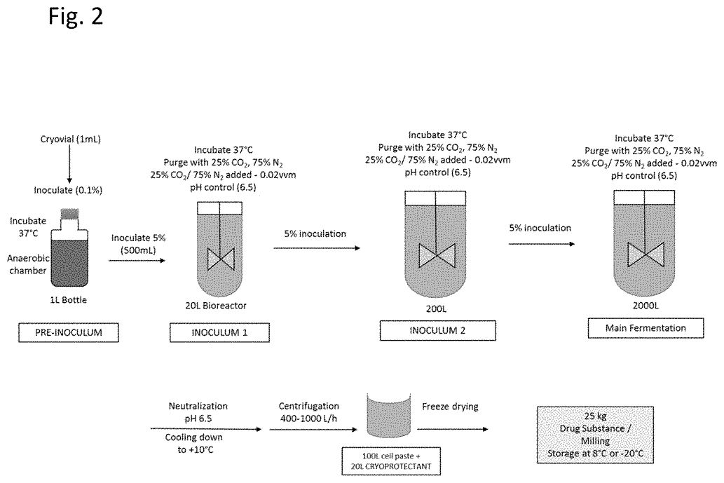

Schematic representations providing exemplary manufacturing methods according to certain embodiments provided herein are depicted in FIGS. 1 and 2.

In certain embodiments, culturing anaerobic bacteria according to a method provided herein results in an improved yield of anaerobic bacteria. In certain embodiments, the yield is improved by a factor of at least 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold or 3.0-fold. In some embodiments, the yield is improved by a factor of between 1.5-fold and 4.0-fold. In some embodiments, the yield is improved by a factor of between 2-fold and 3-fold.

In some embodiments, the methods provided herein reduce contamination of the anaerobic bacteria culture. For example, the methods provided herein can prevent the outgrowth or overgrowth of a contaminant in the anaerobic bacteria culture. Contaminants can include, e.g., bacterial strains present in air flow or gas flow and/or environmental strains, e.g., present at a manufacturing facility.

In certain aspects, provided herein are methods of growing anaerobic bacteria comprising culturing the anaerobic bacteria in a bioreactor under an anaerobic atmosphere comprising CO₂. In some embodiments, the anaerobic atmosphere comprises greater than 1% CO₂. In some embodiments, the anaerobic atmosphere comprises at least about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, about 50%, about 60%, about 70%, about 80%, about 90%, or about 100%, CO₂. In some embodiments, the anaerobic atmosphere comprises greater than 5% CO₂. In some embodiments, the anaerobic atmosphere comprises at least 8% CO₂. In some embodiments, the anaerobic atmosphere comprises at least 10% CO₂. In some embodiments, the anaerobic atmosphere comprises at least 20% CO₂. In some embodiments, the anaerobic atmosphere comprises from 8% to 40% CO₂. In some embodiments, the anaerobic atmosphere comprises from 10% to 40% CO₂. In some embodiments, the anaerobic atmosphere comprises from 20% to 30% CO₂. In some embodiments, the anaerobic atmosphere comprises about 25% CO₂.

In certain aspects, provided herein are methods of growing anaerobic bacteria comprising culturing the anaerobic bacteria in a bioreactor into which an anaerobic gas mixture comprising CO₂ is introduced. In some embodiments, the anaerobic gas mixture comprises greater than 1% CO₂. In some embodiments, the anaerobic gas mixture comprises at least about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, about 50%, about 60%, about 70%, about 80%, about 90%, or about 100%, CO₂. In some embodiments, the anaerobic gas mixture comprises greater than 5% CO₂. In some embodiments, the anaerobic gas mixture comprises at least 8% CO₂. In some embodiments, the anaerobic gas mixture comprises at least 10% CO₂. In some embodiments, the anaerobic gas mixture comprises at least 20% CO₂. In some embodiments, the anaerobic gas mixture comprises from 8% to 40% CO₂. In some embodiments, the anaerobic gas mixture comprises from 10% to 40% CO₂. In some embodiments, the anaerobic gas mixture comprises from 20% to 30% CO₂. In some embodiments, the anaerobic gas mixture comprises about 25% CO₂.

In some embodiments, the anaerobic atmosphere and/or gas mixture comprises less than 95% N₂. In some embodiments, the anaerobic atmosphere and/or gas mixture comprises less than 95%, less than 92%, less than 90%, less than 87%, less than 85%, less than 82%, less than 80%, less than 77% N₂. In some embodiments, the anaerobic atmosphere and/or gas mixture comprises less than 85% N₂. In some embodiments, the anaerobic atmosphere and/or gas mixture comprises less than 80% N₂. In some embodiments, the anaerobic atmosphere and/or gas mixture comprises from 65% to 85% N₂. In some embodiments, the anaerobic atmosphere and/or gas mixture comprises from 70% to 80% N₂. In some embodiments, the anaerobic atmosphere and/or gas mixture comprises about 75% N₂.

In some embodiments, the anaerobic atmosphere and/or gas mixture consists essentially of CO₂ and N₂. In some embodiments, the anaerobic atmosphere and/or gas mixture comprises about 25% CO₂ and about 75% N₂. In some embodiments, the anaerobic atmosphere and/or gas mixture comprises about 20% CO₂ and about 80% N₂. In some embodiments, the anaerobic atmosphere and/or gas mixture comprises about 30% CO₂ and about 70% N₂.

In certain aspects, provided herein are methods of culturing anaerobic bacteria, the method comprises the steps of a) purging a bioreactor with an anaerobic gas mixture comprising greater than 1% CO₂; and b) culturing the anaerobic bacteria in the bioreactor purged in step a). In some embodiments the method comprises introducing the anaerobic gas mixture into the bioreactor during step b). In some embodiments, the anaerobic gas mixture comprises greater than 1% CO₂. In some embodiments, the anaerobic gas mixture comprises at least about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, CO₂. In some embodiments, the anaerobic gas mixture comprises at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, CO₂. In some embodiments, the anaerobic gas mixture comprises from 5% to 35% CO₂, 10% to 40% CO₂, 10% to 30% CO₂, 15% to 30% CO₂, 20% to 30% CO₂, 22% to 28% CO₂, or 24%, to 26% CO₂. In some embodiments, the anaerobic gas mixture comprises greater than 5% CO₂. In some embodiments, the anaerobic gas mixture comprises at least 8% CO₂. In some embodiments, the anaerobic gas mixture comprises at least 10% CO₂. In some embodiments, the anaerobic gas mixture comprises at least 20% CO₂. In some embodiments, the anaerobic gas mixture comprises from 8% to 40% CO₂. In some embodiments, the anaerobic gas mixture comprises from 10% to 40% CO₂. In some embodiments, the anaerobic gas mixture comprises from 20% to 30% CO₂. In some embodiments, the anaerobic gas mixture comprises about 25% CO₂. In some embodiments, CO₂ gas is continuously added during culturing.

In certain aspects, provided herein are methods of culturing anaerobic bacteria, the method comprises the steps of a) purging a bioreactor with an anaerobic gas mixture comprising less than 95% N₂; and b) culturing the anaerobic bacteria in the bioreactor purged in step a). In some embodiments the method comprises introducing the anaerobic gas mixture into the bioreactor during step b). In some embodiments, the anaerobic gas mixture comprises less than 95% N₂. In some embodiments, the anaerobic gas mixture comprises less than 95%, less than 92%, less than 90%, less than 87%, less than 85%, less than 82%, less than 80%, less than 77% N₂. In some embodiments, the anaerobic gas mixture comprises about 65%, about 66%, about 67%, about 28%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the anaerobic gas mixture comprises less than 95% N₂. In some embodiments, the anaerobic gas mixture comprises less than 90% N₂. In some embodiments, the anaerobic gas mixture comprises from 65% to 85% N₂. In some embodiments, the anaerobic gas mixture comprises from 70% to 80% N₂CO₂. In some embodiments, the anaerobic gas mixture comprises about 75% N₂.

In some embodiments, the anaerobic gas mixture consists essentially of CO₂ and N₂. In some embodiments, the anaerobic gas mixture comprises about 25% CO₂ and about 75% N₂. In some embodiments, the anaerobic atmosphere comprises about 20% CO₂ and about 80% N₂. In some embodiments, the anaerobic atmosphere comprises about 30% CO₂ and about 70% N₂.

In some embodiments, the anaerobic gas mixture comprises CO₂ and N₂ in a ratio of about 1:99, about 2:98, about 3:97, about 4:96, about 5:95, about 6:94, about 7:93, about 8:92, about 9:91, about 10:90, 11:89, about 12:88, about 13:87, about 14:86, about 15:85, about 16:84, about 17:83, about 18:82, about 19:81, about 20:80, 21:79, about 22:78, about 23:77, about 24:76, about 25:75, about 26:74, about 27:73, about 28:72, about 29:71, about 30:70, 31:69, about 32:68, about 33:67, about 34:66, about 35:65, about 36:64, about 37:63, about 38:62, about 39:61, or about 40:50 CO₂ to N₂.

In some embodiments, an anaerobic gas mixture is continuously added to the bioreactor during culturing. In some embodiments, the continuously added anaerobic gas mixture is added at a rate of 0.001 to 0.1 vvm. In some embodiments the continuously added anaerobic gas mixture is added at a rate of about 0.001, about 0.002, about 0.003, about 0.004, about 0.005, about 0.006, about 0.007, about 0.008, about 0.009, about 0.01, about 0.011, about 0.012, about 0.013, about 0.014, about 0.015, about 0.016, about 0.017, about 0.018, about 0.019, about 0.02, about 0.03, about 0.04, about 0.05, about 0.06, about 0.07, about 0.08, about 0.09, or about 0.1 vvm. In some embodiments the continuously added anaerobic gas mixture is added at a rate of 0.02 vvm. In some embodiments the continuously added anaerobic gas mixture is added at a rate of about 0.002 vvm. In some embodiments, CO₂ gas is continuously added to the bioreactor during culturing. In some embodiments, the continuously added CO₂ gas is added at a rate of 0.001 to 0.1 vvm. In some embodiments the continuously added CO₂ gas is added at a rate of about 0.001, about 0.002, about 0.003, about 0.004, about 0.005, about 0.006, about 0.007, about 0.008, about 0.009, about 0.01, about 0.011, about 0.012, about 0.013, about 0.014, about 0.015, about 0.016, about 0.017, about 0.018, about 0.019, about 0.02, about 0.03, about 0.04, about 0.05, about 0.06, about 0.07, about 0.08, about 0.09, or about 0.1 vvm. In some embodiments the continuously added a CO₂ gas is added at a rate of about 0.02 vvm. In some embodiments the continuously added CO₂ is added at a rate of about 0.007 vvm. In some embodiments, CO₂ is added at a rate of about 0.1 vvm.

In certain aspects, provided herein are bioreactors comprising anaerobic bacteria under an anaerobic atmosphere comprising at least about 1% CO₂ and/or into which an anaerobic gas mixture comprising at least about 1% CO₂ is added. In some embodiments, the anaerobic atmosphere and/or gas mixture comprises greater than 5% CO₂. In some embodiments, the anaerobic atmosphere and/or gas mixture comprises at least 8% CO₂. In some embodiments, the anaerobic atmosphere and/or gas mixture comprises at least 20% CO₂. In some embodiments, the anaerobic atmosphere and/or gas mixture comprises from 8% to 40% CO₂. In some embodiments, the anaerobic atmosphere and/or gas mixture comprises from 10% to 40% CO₂. In some embodiments, the anaerobic atmosphere and/or gas mixture comprises from 20% to 30% CO₂. In some embodiments, the anaerobic atmosphere and/or gas mixture comprises about 25% CO₂.

In certain aspects, provided herein are bioreactors comprising anaerobic bacteria under an anaerobic atmosphere comprising less than 95% N₂ and/or into which an anaerobic gas mixture comprising less than 95% N₂ is added. In some embodiments, the anaerobic atmosphere and/or gas mixture comprises less than 90% N₂. In some embodiments, the anaerobic atmosphere and/or gas mixture comprises less than 85% N₂. In some embodiments, the anaerobic atmosphere and/or gas mixture comprises from 65% to 85% N₂. In some embodiments, the anaerobic atmosphere and/or gas mixture comprises from 70% to 80% N₂. In some embodiments, the anaerobic atmosphere and/or gas mixture comprises about 75% N₂.

In some embodiments, the anaerobic atmosphere and/or gas mixture consists essentially of CO₂ and N₂. In some embodiments, the anaerobic atmosphere and/or gas mixture comprises about 25% CO₂ and about 75% N₂. In some embodiments, the anaerobic atmosphere and/or gas mixture comprises about 20% CO₂ and about 80% N₂. In some embodiments, the anaerobic atmosphere and/or gas mixture comprises about 30% CO₂ and about 70% N₂. In some embodiments, the bioreactor is at least 1 L, 20 L, 3500 L, 20,000 L, 50,000 L, 100,000 L, 200,000 L, 300,000 L, 400,000 L or 500,000 L.

In certain aspects, provided herein are methods of growing anaerobic bacteria comprising culturing the anaerobic bacteria in a bioreactor comprising carbonate salt. In some embodiments, is sodium carbonate, potassium carbonate, barium carbonate, carbonic acid, magnesite (magnesium carbonate), sodium percarbonate (adduct with hydrogen peroxide), or calcium carbonate. In some embodiments, the carbonate is at a concentration of 0.5 g/L to 10 g/L. In some embodiments, the carbonate salt is at a concentration of 0.5 to 1 g/L, 1 to 5 g/L, or 2 to 8 g/L. In some embodiments, the carbonate salt is at a concentration of about 0.5 g/L, about 1 g/L, about 5 g/L, or about 10 g/L. In some embodiments, the bioreactor is at least 1 L, 20 L, 3500 L, 20,000 L, 50,000 L, 100,000 L, 200,000 L, 300,000 L, 400,000 L or 500,000 L.

In certain aspects, provided herein are methods of growing anaerobic bacteria comprising culturing the anaerobic bacteria in a bioreactor comprising bicarbonate salt. In some embodiments, the bicarbonate salt is sodium bicarbonate, potassium bicarbonate, cesium bicarbonate, magnesium bicarbonate, or ammonium bicarbonate. In some embodiments, the bicarbonate salt is at a concentration of 0.5 g/L to 10 g/L. In some embodiments, the bicarbonate salt is at a concentration of 0.5 to 1 g/L, 1 to 5 g/L, or 2 to 8 g/L. In some embodiments, the bicarbonate salt is at a concentration of about 0.5 g/L, about 1 g/L, about 5 g/L, or about 10 g/L. In some embodiments, the bioreactor is at least 1 L, 20 L, 3500 L, 20,000 L, 50,000 L, 100,000 L, 200,000 L, 300,000 L, 400,000 L or 500,000 L.

In some embodiments, the methods and compositions provided herein include the culturing of anaerobic bacteria in growth media. In some embodiments the growth media may contain sugar, yeast extracts, plant based peptones, buffers, salts, trace elements, surfactants, anti-foaming agents, and/or vitamins.

The sugar source present in the growth media can affect growth. For example, use of maltodextrin (e.g., glucidex, e.g., glucidex 21D) can provide better growth than use of glucose, e.g., at the same concentration of each sugar source, e.g., about 10 g/L. Alternatively, maltodextrin and glucose can both be used in the growth media, e.g., glucose at 10 g/L and maltodextrin at 25 g/L. For example, use of maltodextrin (e.g., glucidex, e.g., glucidex 21D) and glucose can provide better growth than use of glucose alone, e.g., at the same total concentration, e.g., about 35 g/L total sugar. In some embodiments, the growth media comprises glucose. In some embodiments, the growth media comprises maltodextrin. In some embodiments, the growth media comprises glucose and maltodextrin.

In some embodiments, the growth media comprises yeast extract, soy peptone A2SC 19649, Soy peptone E110 19885, dipotassium phosphate, monopotassium phosphate, L-cysteine-HCl, ammonium chloride, maltodextrin (e.g., glucidex, e.g., glucidex 21D), glucose, and/or hemoglobin. In some embodiments, the growth media comprises 5 g/L to 15 g/L yeast extract 19512. In some embodiments, the growth media comprises 10 g/L yeast extract 19512. In some embodiments, the growth media comprises 10 g/L to 15 g/L soy peptone A2SC 19649. In some embodiments, the growth media comprises 12.5 g/L soy peptone A2SC 19649. In some embodiments, the growth media comprises 10 g/L to 15 g/L Soy peptone E110 19885. In some embodiments, the growth media comprises 12.5 g/L Soy peptone E110 19885. In some embodiments, the growth media comprises 1 g/L to 2 g/L dipotassium phosphate. In some embodiments, the growth media comprises 1.59 g/L Dipotassium phosphate. In some embodiments, the growth media comprises 0.5 g/L to 1.5 g/L monopotassium phosphate. In some embodiments, the growth media comprises 0.91 g/L monopotassium phosphate. In some embodiments, the growth media comprises 0.1 g/L to 1.0 g/L L-cysteine-HCl. In some embodiments, the growth media comprises 0.5 g/L L-cysteine-HCl. In some embodiments, the growth media comprises 0.1 g/L to 1.0 g/L ammonium chloride. In some embodiments, the growth media comprises 0.5 g/L ammonium chloride. In some embodiments, the growth media comprises 20 g/L to 30 g/L maltodextrin (e.g., glucidex, e.g., glucidex 21D). In some embodiments, the growth media comprises 25 g/L maltodextrin (e.g., glucidex, e.g., glucidex 21D). In some embodiments, the growth media comprises 5 g/L to 15 g/L glucose. In some embodiments, the growth media comprises 10 g/L glucose. In some embodiments, the growth media comprises 0.01 g/L to 0.05 g/L hemoglobin. In some embodiments, the growth media comprises 0.02 g/L hemoglobin.

In some embodiments, the growth media comprises yeast extract, soy peptone A2SC 19649, Soy peptone E110 19885, dipotassium phosphate, monopotassium phosphate, L-cysteine-HCl, ammonium chloride, glucose, and/or hemoglobin. In some embodiments, the growth media comprises 5 g/L to 15 g/L yeast extract 19512. In some embodiments, the growth media comprises 10 g/L yeast extract 19512. In some embodiments, the growth media comprises 10 g/L to 15 g/L soy peptone A2SC 19649. In some embodiments, the growth media comprises 12.5 g/L soy peptone A2SC 19649. In some embodiments, the growth media comprises 10 g/L to 15 g/L Soy peptone E110 19885. In some embodiments, the growth media comprises 12.5 g/L Soy peptone E110 19885. In some embodiments, the growth media comprises 1 g/L to 2 g/L dipotassium phosphate. In some embodiments, the growth media comprises 1.59 g/L Dipotassium phosphate. In some embodiments, the growth media comprises 0.5 g/L to 1.5 g/L monopotassium phosphate. In some embodiments, the growth media comprises 0.91 g/L monopotassium phosphate. In some embodiments, the growth media comprises 0.1 g/L to 1.0 g/L L-cysteine-HCl. In some embodiments, the growth media comprises 0.5 g/L L-cysteine-HCl. In some embodiments, the growth media comprises 0.1 g/L to 1.0 g/L ammonium chloride. In some embodiments, the growth media comprises 0.5 g/L ammonium chloride. In some embodiments, the growth media comprises 5 g/L to 15 g/L glucose. In some embodiments, the growth media comprises 10 g/L glucose. In some embodiments, the growth media comprises 0.01 g/L to 0.05 g/L hemoglobin. In some embodiments, the growth media comprises 0.02 g/L hemoglobin.

In some embodiments, the growth media comprises yeast extract, soy peptone A2SC 19649, Soy peptone E110 19885, dipotassium phosphate, monopotassium phosphate, L-cysteine-HCl, ammonium chloride, maltodextrin (e.g., glucidex, e.g., glucidex 21D), and/or hemoglobin. In some embodiments, the growth media comprises 5 g/L to 15 g/L yeast extract 19512. In some embodiments, the growth media comprises 10 g/L yeast extract 19512. In some embodiments, the growth media comprises 10 g/L to 15 g/L soy peptone A2SC 19649. In some embodiments, the growth media comprises 12.5 g/L soy peptone A2SC 19649. In some embodiments, the growth media comprises 10 g/L to 15 g/L Soy peptone E110 19885. In some embodiments, the growth media comprises 12.5 g/L Soy peptone E110 19885. In some embodiments, the growth media comprises 1 g/L to 2 g/L dipotassium phosphate. In some embodiments, the growth media comprises 1.59 g/L Dipotassium phosphate. In some embodiments, the growth media comprises 0.5 g/L to 1.5 g/L monopotassium phosphate. In some embodiments, the growth media comprises 0.91 g/L monopotassium phosphate. In some embodiments, the growth media comprises 0.1 g/L to 1.0 g/L L-cysteine-HCl. In some embodiments, the growth media comprises 0.5 g/L L-cysteine-HCl. In some embodiments, the growth media comprises 0.1 g/L to 1.0 g/L ammonium chloride. In some embodiments, the growth media comprises 0.5 g/L ammonium chloride. In some embodiments, the growth media comprises 20 g/L to 30 g/L maltodextrin (e.g., glucidex, e.g., glucidex 21D). In some embodiments, the growth media comprises 25 g/L maltodextrin (e.g., glucidex, e.g., glucidex 21D). In some embodiments, the growth media comprises 0.01 g/L to 0.05 g/L hemoglobin. In some embodiments, the growth media comprises 0.02 g/L hemoglobin.

In some embodiments, the media is sterilized. Sterilization may be by Ultra High Temperature (UHT) processing, autoclaving or filtering. The UHT processing is performed at very high temperature for short periods of time. The UHT range may be from 135-180° C. For example, the medium may be sterilized from between 10 to 30 seconds at 135° C.

In some embodiments, inoculum can be prepared in flasks or in smaller bioreactors where growth is monitored. For example, the inoculum size may be between approximately 0.1% v/v and 5% v/v of the total bioreactor volume. In some embodiments, the inoculum is 0.1-3% v/v, 0.1-1% v/v, 0.1-0.5% v/v, or 0.5-1% v/v of the total bioreactor volume. In some embodiments, the inoculum is 0.1% v/v, 0.2% v/v, 0.3% v/v, 0.4%, v/v, 0.5% v/v, 0.6% v/v, 0.7% v/v, 0.8% v/v, 0.9% v/v, 1% v/v, 1.5% v/v, 2% v/v, 2.5% v/v, 3% v/v 4%, v/v, or 5% v/v of the total bioreactor volume.

Depending on the application and need for material, bioreactor volume can be at least 1 L, 2 L, 10 L, 80 L, 100 L, 250 L, 1000 L, 2500 L, 3500 L, 5000 L, 10,000 L, 20,000 L, 50,000 L, 100,000 L, 200,000 L, 300,000 L, 400,000 L or 500,000 L.

In some embodiments, before the inoculation, the bioreactor is prepared with growth medium at desired pH and temperature. The initial pH of the culture medium may be different that the process set-point. pH stress may be detrimental at low cell centration; the initial pH could be between pH 7.5 and the process set-point. For example, pH may be set between 4.5 and 8.0, preferably 6.5. During the fermentation, the pH can be controlled through the use of sodium hydroxide, potassium hydroxide, or ammonium hydroxide. The temperature may be controlled from 25° C. to 45° C., for example at 37° C.

In certain embodiments, anaerobic conditions are created by reducing the level of oxygen in the bioreactor by introducing or purging the bioreactor with nitrogen, carbon dioxide or gas mixtures (N₂ and CO₂) in order to establish an anaerobic atmosphere in the bioreactor.

In some embodiments, the atmosphere comprises at least about 2% to about 40% CO₂, about 5% to 35% CO₂, about 10% to 30% CO₂, about 15% to 30% CO₂, about 20% to 30% CO₂, about 22% to 28% CO₂, or about 24% to 26% CO₂. In some embodiments, the anaerobic gas mixture comprises greater than 5% CO₂. In some preferred embodiments, the atmosphere comprises about 25% CO₂.

In some embodiments, the atmosphere comprises at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% CO₂. In some embodiments, the anaerobic gas mixture comprises greater than 5% CO₂. In some preferred embodiments, the atmosphere comprises at least about 25% CO₂.

In some embodiments, the atmosphere comprises 65% to 85% N₂ or 70% to 80% N₂. In some embodiments, the anaerobic gas mixture comprises less than 95% N₂. In some preferred embodiments, the atmosphere comprises about 75% N₂.

In some embodiments, the atmosphere comprises about 65%, about 66%, about 67%, about 28%, about 69%, about 70%, about 71%, about 72% about 73%, about 74%, about 75%, about 76%, about 77%, about 78%, about 79%, about 80%, about 81%, about 82%, about 83%, about 84%, about 85% N₂. In some embodiments, the anaerobic gas mixture comprises less than 95% N₂. In some preferred embodiments, the atmosphere comprises about 75% N₂.

In some embodiments, the gas mixture (CO₂ and N₂) provides an atmosphere in the bioreactor comprising CO₂ and N₂ in a ratio of about 1:99, about 2:98, about 3:97, about 4:96, about 5:95, about 6:94, about 7:93, about 8:92, about 9:91, about 10:90, 11:89, about 12:88, about 13:87, about 14:86, about 15:85, about 16:84, about 17:83, about 18:82, about 19:81, about 20:80, 21:79, about 22:78, about 23:77, about 24:76, about 25:75, about 26:74, about 27:73, about 28:72, about 29:71, about 30:70, 31:69, about 32:68, about 33:67, about 34:66, about 35:65, about 36:64, about 37:63, about 38:62, about 39:61, or about 40:50 CO₂ to N₂. In some embodiments, the mixed gas composition provides an atmosphere in the bioreactor comprising CO₂ and N₂ in a ratio of about 25:75.

In some embodiments, depending on strain and inoculum size, the bioreactor fermentation time can vary. For example, fermentation time can vary from approximately 5 hours to 48 hours. In some embodiments, fermentation time may be from about 5 hours to about 24 hours, about 8 hours to about 24 hours, about 8 hours to about 18 hours, about 8 hours to about 16 hours, about 8 hours to about 14 hours, about 10 hours to about 24 hours, about 10 hours to about 18 hours, about 10 hours to about 16 hours, about 10 hours to about 14 hours, about 10 hours to about 12 hours, about 12 hours to about 24 hours, about 12 hours to about 18 hours, about 12 hours to about 16 hours, or about 12 hours to about 14 hours. In some embodiments, fermentation time may be from about 12 hours to about 96 hours, from about 12 hours to about 72 hours, from about 12 hours to about 60 hours, from about 24 hours to about 96 hours, from about 24 hours to about 72 hours, from about 24 hours to about 60 hours, from about 24 hours to about 48 hours, from about 36 hours to about 96 hours, from about 36 hours to about 72 hours, from about 36 hours to about 60 hours, or from about 36 hours to about 48 hours.

In some embodiments, fermentation culture is continuously mixed with addition of a mixed gas composition of CO₂ and N₂. In some embodiments, the mixed gas composition provides an atmosphere in the bioreactor comprising CO₂ and N₂ in a ratio of about 1:99, about 2:98, about 3:97, about 4:96, about 5:95, about 6:94, about 7:93, about 8:92, about 9:91, about 10:90, 11:89, about 12:88, about 13:87, about 14:86, about 15:85, about 16:84, about 17:83, about 18:82, about 19:81, about 20:80, 21:79, about 22:78, about 23:77, about 24:76, about 25:75, about 26:74, about 27:73, about 28:72, about 29:71, about 30:70, 31:69, about 32:68, about 33:67, about 34:66, about 35:65, about 36:64, about 37:63, about 38:62, about 39:61, or about 40:50 CO₂ to N₂. In some embodiments, the mixed gas composition provides an atmosphere in the bioreactor comprising CO₂ and N₂ in a ratio of about 25:75.

In certain embodiments, harvest time may be based on either glucose level is below 2 g/L or when stationary phase is reached.

In some embodiments, once fermentation complete, the culture is cooled (e.g., to 10° C.) and centrifuged collecting the cell paste. A stabilizer may be added to the cell paste and mixed thoroughly. Harvesting may be performed by continuous centrifugation. Product may be resuspended with various excipients to a desired final concentration. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets may be mixed with excipients and submerged in liquid nitrogen.

In certain embodiments, the cell slurry may be lyophilized. Lyophilization of material, including live bacteria, may begin with primary drying. During the primary drying phase, the ice is removed. Here, a vacuum is generated and an appropriate amount of heat is supplied to the material for the ice to sublime. During the secondary drying phase, product bound water molecules may be removed. Here, the temperature is raised higher than in the primary drying phase to break any physico-chemical interactions that have formed between the water molecules and the product material. The pressure may also be lowered further to enhance desorption during this stage. After the freeze-drying process is complete, the chamber may be filled with an inert gas, such as nitrogen. The product may be sealed within the freeze dryer under dry conditions, preventing exposure to atmospheric water and contaminants. The lyophilized material may be gamma irradiated (e.g., 17.5 kGy).

### Anaerobic Bacteria

In some aspects, provided herein are methods and compositions for culturing anaerobic bacteria. In certain aspects, the anaerobic bacteria used in the methods and compositions provided herein are selected from bacteria of the genus Actinomyces, Bacteroides, Clostridium, Fusobacterium, Peptostreptococcus, Porphyromonas, Prevotella, Propionibacterium, or Veillonella.

In some embodiments, the anaerobic bacteria are Prevotella bacteria of the species Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella melanogenica, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

In some embodiments, the Prevotella is Prevotella Strain B 50329 (NRRL accession number B 50329). In some embodiments, the Prevotella strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

In some embodiments, the Prevotella bacteria is a strain of Prevotella bacteria comprising one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) proteins listed in Table 1 and/or one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) genes encoding proteins listed in Table 1. In some embodiments, the Prevotella bacteria comprises all of the proteins listed in Table 1 and/or all of the genes encoding the proteins listed in Table 1.

TABLE 1
Exemplary Prevotella proteins

| Seq. ID. No. | Name | Uniprot ID | Amino Acid Sequence |
|---|---|---|---|
| 1 | Cluster: | G6ADE1 | MNLKTFTKTVLCFALFAVSAITAKAADHLAIVGE |
| | Uncharacterized | | AVWGGWDLVKATAMVKSPNNPDVFMATVHLNAGK |
| | protein | | GFKFLTEREWGKLEYRSGASDVVLKSGIRYKLYA |
| | | | SIGASEDGKFKVSESANYEIICDLARKTVEVKKV |
| | | | AYQAKEIRYAALWMIGDATAGDWDYNNGVLLSQD |
| | | | SGNPTCYTATVELKEGEFKFTTNKQWGYDHSVYI |
| | | | FRDVNDQNKIVFGGEDNKWRITEDGMYNVTVDVP |
| | | | TKTISIKQIDDPAGHKPQFGNDVILVGDATIAGW |
| | | | NLDNAIYLEHTGQAGRVFKTTTYLEAGKGFKFLS |
| | | | MLSYDDIDYRPANNTVLNPGVPGTFVPSLPSSTD |
| | | | TKFSVERSGNYDIVCNMNNRTVVVTLSENQVLVN |
| | | | YPALWLIGSATSAGWNPGKAVELKRSEADPAVYT |
| | | | ARVQLKKGEFKILTSKNVGFDQPTYYRDSTNEHR |
| | | | IVFGVDGDEVAKKDCKWTLSENAEGTYDVTVDIE |
| | | | AMTIFCDKVNMDEPSVESTDKELILIGDATYSAW |
| | | | DLPKSIVMTPVGPTTFKAVTHLEAGKEFKFLTEL |
| | | | AWKRYEYRAESLRKELQEGSMSMLVPYRYTNDKD |
| | | | DKDHDFKFVVKESGNYEIVCDLYIPALIIRKVRY |
| | | | QDTPVTYSSLWIVGSATPGGWTIERGIKMTQDEN |
| | | | YPTKFTAKANLVPGELKFATNKFADFTQDFFFRG |
| | | | KDDYTAVLGGNDNKWNITEAGTYSVTIDVASKRV |
| | | | TITKPARNAPTGISTVDSSDEAPAEYFTLNGIKV |
| | | | TTPSSGIYIKRQGGRTTKVVMK |
| 2 | Nicotinamide_ | P24520 | MDTYQILDIIGCIVGLIYIYQEYKASIWLWMTGI |
| | riboside_ | | IMPVIYMFVYYEAGLYADFGMQIYYTLAAIYGYL |
| | transporter_PnuC | | YWKLGKKKGTEDKEIPITHFPRRYIIPAIIVFFV |
| | | | LWIALYYILICFTNSTVPVLDSFGNALSFIGLWA |
| | | | LAKKYLEQWWIWIVVDAELSALYIYKGIPFTAML |
| | | | YALYTVIAVAGYFKWRRYIKQQK |
| 3 | Pectate_trisaccharide- | Q8GCB2 | MRVRLYKNILLFLFLWVNTLACVSADTSRTVESQ |
| | lyase | | PIENGLIITESKGWLETIYAKWKPVAEADGYYVY |
| | | | VKGGQYADYSKVDSELIRVYNGYVRVDIPGLKAG |
| | | | TYSLKIVAVKGGKETQSSEVTGLKVLNYVREGFA |
| | | | HKNYSGVGAYNDDGTLKSGAVVIYVNKDNAKTVS |
| | | | AHLGKTTFIGLQAILNAYQKGNITTPLSVRILGL |
| | | | LRNGDTDTFGSSTEGIQIKGKQADSEMNITIEGI |
| | | | GEDASIYGFGFLVRNAKSVEFRNLGIMRAMDDGV |
| | | | SLDTNNSNIWIHHMDLFYGKASGGDHIKGDGSID |
| | | | VKTDSKYVTIDNCHFWDTGKTSMCGMKKETGPNY |
| | | | ITYHHNWFDHSDSRHARVRTMSVHLWNNYYDGCA |
| | | | KYGIGATMGCSVFSENNYFRATKNPILISKQGSD |
| | | | AKGTGKFSGEPGGMVKEYGSLFTEKGAESTYTPI |
| | | | SYADNNSSFDFYHAISRNEKVPASVKTLNGGNIY |
| | | | NNFDTDAALMYSYTPDATALVPSQVTGFYGAGRL |
| | | | NHGSLQFKFNNAVEDTNSTPIPALEALIDAYSGK |
| 4 | Glycosyltransferase_ | Q9AET5 | MKYNIAYCIEGFYNHGGMERILSVCANLLSDIYS |
| | Gtf1 | | ITIIVANQRGREHAYNLAQNVNVVDLGVSCKNYK |
| | | | EEYKKSLTRYLQDHQFSVVISLAGLELFFLPQIK |
| | | | DGSKKVMWFHFAFDVSKMFLSERFHGWKLNLLYY |
| | | | IHTIRRIYFAKKFDTIVVLSKSDCDSWSRFCNNV |
| | | | KYIYNPITIDRKVISNLSEESVIAVGRLGWQKGF |
| | | | DFLIDSWVLVDDKHPDWHLDIFGEGPDRLELQHQ |
| | | | IDRKGLHDKVRLCGVTKQIEEEYGKHSIYVMSSR |
| | | | AEGFPLALLEASSCGLPMISFNCHQGPNEIIQEG |
| | | | ENGFLVDKVGDIYTLSDRICKLIEDNNLRNMMGK |
| | | | KALDSSFRFEGEVIKKDWISLLKQLI |
| 5 | Cluster: Protein | A0A096B759 | MKRLFFMFLFLGTITMNSLAQEEKPIKYETKNFS |
| | TonB | | LPDKMPLYPGGDGALRAFLSLNLHYPEKAQAFGV |
| | | | EGRSLMKFCVSSDGSIKDISAVDCKITNYNRTEF |
| | | | NKLPLSKQESLKKECAKAFAKEAARVIRLMPKWE |
| | | | PAELNGKKMNVYYSLPFTFKLR |
| 6 | Cluster: | G6AEN6 | MNYPLFIARKIYNGGDRTRKVSKPAIRIATIGVA |
| | Uncharacterized | | IGLAVMIISVGVVLGFKHTIRNKVVGFGSDITVA |
| | protein | | NFLTLQSSEQYPIQITDSLVKSLQITPGIKHVQR |
| | | | YDYTQGILKTDNDFLGVLLKGVGPDFDSTFIHEN |
| | | | MVEGSLPHFHDNESQQKIVISKTIADKLNLKVGQ |
| | | | RIFAYFINKQGVRTRKFTITGIYATNMKQFDSQI |
| | | | CFTDIYTTNKLNGWEPDQYSGAELQVDNFSQLTP |
| | | | ISMRVLNKVKNTVDHYGGTYSSENIIEQNPQIFS |
| | | | WLDLMDMNVWIILALMISVAGVTMISGLLIIILE |
| | | | RTQMIGILKALGSRNRQIRHIFLWFATFIIGKGL |
| | | | LWGNIIGLGCILFQSWTGLVKLDPQTYYVNTVPV |
| | | | EINIPLIIALNMVTMLVCLVILIAPSYLISHIHP |
| | | | AKSMHYE |
| 7 | Bifunctional_ | P9WHG9 | MEDKFIYTDKERKLSYQILDELKDTLDKSFLEND |
| | (p)ppGpp_synthase/ | | LPMLQVQLKDSVAKNTIHRNVFGLNPILCSLQTA |
| | hydrolase_RelA | | AIAVKDIGLKRDSVIAILLHQSVQDGYITLEDID |
| | | | NRFGKSVAKIIHGLIRIQTLYQKNPIIESENFRN |
| | | | LLLSFAEDMRVILIMIADRVNLMRQIRDAEDKEA |
| | | | QHKVAEEASYLYAPLAHKLGLYQLKRELEDLSLK |
| | | | YLEHDAYYLIKDKLNATKASRDAYINQFIAPVRE |
| | | | RLTAGGLRFHIKGRTKSIHSIWQKMKKQKCGFEG |
| | | | IYDLFAIRIILDAPLEKEKIQCWQAYSIVTDMYQ |
| | | | PNPKRLRDWLSVPKSNGYECLHITVLGPEKKWVE |
| | | | VQIRTERMDEIAEHGLAAHWRYKGIKEEGGLDDW |
| | | | LASIRAALEAGDNLEVMDQFKSDLYEKEIYVFTP |
| | | | KGDLLKFPKGATILDFAYHIHSKVGNQCVGGKIN |
| | | | AKNVSLRTELHSGDTVEILTSATQKPKAEWLKIV |
| | | | KSSRAKAKIRLALKETQIKDGLYAKELLERRFKN |
| | | | KKIEIEESTMGHLLRKLGFKEVSEFYKQVADEKL |
| | | | DPNYIIEEYQKVYNHDHNLNQPKETESAENFEFE |
| | | | NPTNEFLKKNDDVLVIDKNLKGLDFSLAKCCHPI |
| | | | YGDPVFGFVTVNGGIKIHRTDCPNAPEMRKRFGY |
| | | | RIVKARWSGKGSSQYAITLRVIGNDDIGIVSNIT |
| | | | NVISKDEKIVMRSINIDSHDGLFSGNLVVLLDDN |
| | | | SKLNMLIKKLRTVKGVKQVTRI |
| 8 | Vitamin_B12_ | P06609 | MKRRIFLFVALSVSIVILFGLNLIIGSVHIPLSD |
| | import_system_ | | ILTILSGSFTGKESWRFIIWDSRLPQALTAMLCG |
| | permease_protein_BtuC | | SSLAVCGLMLQTAFRNPLAGPDVFGISSGASLGV |
| | | | ALVMLLLGGTVETSMFTASGFLAILIVAFAGAIL |
| | | | VTAFILFLSSVVRNSVLLLIVGIMVGYVASSAVT |
| | | | LLNFFSSEDGVKGYIVWGMGNFGGVSMSHIPLFA |
| | | | FLCLAGIIASFLLVKPLNILLLGPQYAESLGISI |
| | | | RRIRNILLVVVGILTAVTTAFCGPISFIGLAAPH |
| | | | VARLLFRTENHQKLLPGTLLVGTVVALLCNLICF |
| | | | LPRESGMIPLNAVTPLIGAPIIIYVIMKRH |
| 9 | NADH- | P33599 | MKLENKEFGFDSFATEMARLKNEKHFDYLVTVVG |
| | quinone_ | | EDFGTEEGLGCIYILENTSTHERCSVKQLAKKVG |
| | oxidoreductase_ | | EEFVIPSVIKLWADADLLEREVYDFYGIKFLGHP |
| | subunit_C/D | | DMRRLFLRNDFKGYPLRKDYDMDPAKNMYTTEDD |
| | | | VELDTTTEWNLDKNGELVGTQHALFTDDNFVVNI |
| | | | GPQHPSTHGVLRLQTVLDGETVTNIYPHLGYIHR |
| | | | GIEKLCEQFTYPQTLALTDRMNYLSAMMNRHALV |
| | | | GVIEEGMGIELSERILYIRTIMDELQRIDNHLLY |
| | | | TACCAQDLGALTAFLYGMRDREHVLNVMEETTGG |
| | | | RLIQNYYRIGGLQADIDPNFVSNVKELCKYLRPM |
| | | | IQEYVDVFGDNVITHQRFEGVGVMDEKDCISYGV |
| | | | TGPAGRASGWKNDVRKYHPYAMYDKVNFEEITLT |
| | | | NGDSMDRYFCHIKEIYQSLNIIEQLIDNIPEGEF |
| | | | YIKQKPIIKVPEGQWYFSVEGASGEFGAYLDSRG |
| | | | DKTAYRLKFRPMGLTLVGAMDKMLRGQKIADLVT |
| | | | TGAALDFVIPDIDR |
| 10 | FKBP- | P45523 | MRTSTQSKDMGKKQEYKLRNEEFLHNISKKDSIK |
| | type_peptidyl- | | TLPHGIFYEIIKEGSGEGTVQPRSIVICNYRGSL |
| | prolyl_cis- | | ISGQVFDDSWQKPTPEAFRLNELITGLQIALCAM |
| | trans_isomerase | | HKGDSWRIYIPYQEGYGSKRNADIPAFSTLIFDI |
| | | | ELINIA |
| 11 | Putative_acetolactate_ | P9WKJ3 | MADNKIAKESVKREVIAGERLYTLLVYSENVAGV |
| | synthase_small_ | | LNQIAAVFTRRQVNIESLNVSASSIEGIHKYTIT |
| | subunit | | AWSDAATIEKITKQVEKKIDVIKADYYEDSDLFI |
| | | | HEVGLYKIATPILLENAEVSRAIRKRNARMMEVN |
| | | | PTYSTVLLAGMTDEVTALYHDLKNFDCLLQYSRS |
| | | | GRVAVTRGFSEPVSDFLKSEEESSVL |
| 12 | Serine/threonine_ | P0AGE4 | MKKKVKIGLLPRVIIAILLGIFFGYFMPTPLARV |
| | transporter_SstT | | FLTFNGIFSQFLGFMIPLIIIGLVTPAIADIGKG |
| | | | AGKLLLVTVIIAYVDTVVAGGLAYGTGLCLFPSM |
| | | | IASTGGAMPHIDKATELAPYFSINIPAMADVMSG |
| | | | LVFSFMLGLGIAYGGLTATKNIFNEFKYVIEKVI |
| | | | AKAIIPLLPLYIFGVFLNMAHNGQAQQILLVFSQ |
| | | | IIIVILVLHVFILVYQFCIAGAIIRRNPFRLLWN |
| | | | MMPAYLTALGTSSSAATIPVTLEQTMKNGVGKEI |
| | | | AGFVVPLCATIHLSGSAMKITACALTICLLVGLP |
| | | | HDPALFIYFILMLSIIMVAAPGVPGGAIMAALAP |
| | | | LASILGFNSEAQALMIALYIAMDSFGTACNVTGD |
| | | | GAIALVVNKMFGKKER |
| 13 | Cluster: | G6AJ07 | MKKLLLLVCAAVMSLSASAQAGDKALGAQLVFGS |
| | Uncharacterized | | ETNSLGFGVKGQYYFTDHIRGEGSFDYFLKNKGI |
| | protein | | SMWDINANVHYLFDVADKFKVYPLAGLGYTNWSY |
| | | | KYEYAGAPVVEGSDGRLAVNLGGGVEYELTKNLN |
| | | | VNAEAKYQIISNYNQLVLGVGVAYKF |
| 14 | Heterocyst_ | P22638 | MHFYCTKSSLDTMSERYVKRMIAKLASQGKTVIS |
| | differentiation_ATP- | | IAHRFSTIMDAKHIILLAKGKVVAEGTHQELLKT |
| | binding_protein | | SEDYRKLWSDQNDEID |
| 15 | UDP-2,3- | Q9I2V0 | MKNVYFLSDAHLGSLAIAHRRTQERRLVRFLDSI |
| | diacylglucosamine_ | | KHKASAVYLLGDMFDFWDEYKYVVPKGFTRFLGK |
| | hydrolase | | VSELTDMGVEVHFFTGNHDLWTYGYLEEECGVIL |
| | | | HRKPVTMEIYGKVFYLAHGDGLGDPDPMFQFLRK |
| | | | VFHNRVCQRLLNFFHPWWGMQLGLNWAKKSRLKR |
| | | | ADGKEMPYLGEDKEYLVRYTKDYMRSHKDIDYYI |
| | | | YGHRHIELDLTLSGKVRMLILGDWIWQFTYAVFD |
| | | | GEHMFLEEYIEGESKP |
| 16 | Anaerobic_glycerol- | P0A9C0 | MNSKQNDNYDVIIIGGGITGAGTARDCALRGLKV |
| | 3-phosphate_ | | LLVEKFDFTNGATGRNHGLLHSGARYAVTDPESA |
| | dehydrogenase | | TECIKENMVLRRIAKHCIEETDGLFITLPEDDIN |
| | | | YQKTFVEACARAGISANIISPEEALRLDPSVNPD |
| | | | LLGAVRVPDASVDPFHLTTANVLDARQHGADVLT |
| | | | YHEVVAILTSNGRVEGVRLRNNHTGEEIEKHAVL |
| | | | VINAAGIWGHDIAKMADIKINMFPAKGTLLVFGH |
| | | | RVNKMVINRCRKPANADILVPDDAVCVIGTTSDR |
| | | | VPYDTVDNLKITSEEVDTLIREGEKLAPSLATTR |
| | | | ILRAYAGVRPLVAADNDPTGRSISRGIVCLDHEK |
| | | | RDGLTGMITITGGKMMTYRLMAEQATDLACKKLG |
| | | | INKTCETATTPLPGTAGKDSDNPHHTYSTAHKAA |
| | | | KGRQGNRVKEIDERTEDDRALICECEEVSVGEAK |
| | | | YAIEELHVHDLLNLRRRTRVGMGTCQGELCACRA |
| | | | AGVMCENGVKVDKAMTDLTKFINERWKGMRPVAW |
| | | | GSTLDEAQLTTIIYQGLCGLGI |
| 17 | Anaerobic_glycerol- | P13033 | MRYDTIIIGGGLSGLTAGITLAKAGQKVCIVSAG |
| | 3-phosphate_ | | QSSLHFHSGSFDLLGYDADGEVVTHPLQAIADLK |
| | dehydrogenase | | AEHPYSKIGISNIEHLASQAKTLLCEAGISVMGN |
| | | | YEQNHYRVTPLGTLKPAWLTTEGYAMIDDPEILP |
| | | | WKKVELLNIQGFMDFPTQFIAENLRMMGVECQIK |
| | | | TFTTDELSTARQSPTEMRATNIAKVLANKDALSK |
| | | | VSERINAISGDPDALLLPAVLGFSNAESLDEMKQ |
| | | | WIKKPVQYIATLPPSVSGVRTTILLKRLFAQAGG |
| | | | TLLIGDSATTGQFSGNHLVSITTDHLPDEKLYAD |
| | | | HFILASGSFMSHGIRSNYAGVYEPVFKLDVDAAE |
| | | | KRDDWSVTNAFEAQPYMEFGVHTDKDFHATKDGK |
| | | | NIENLYAIGSVLSGHNSIKHADGTGVSLLTALYV |
| | | | AKKITGKG |
| 18 | Anaerobic_glycerol- | P0A996 | MAEGIQLKNISGNNLEQCLKCSICTAYCPVSAVE |
| | 3-phosphate_ | | PKYPGPKQSGPDQERYRLKDSKFFDEALKMCLNC |
| | dehydrogenase | | KRCEVACPSGVRIADIIQASRITYSTHRPIPRDI |
| | | | MLANTDFVGTMANMVAPIVNATLGLKPVKAVLHG |
| | | | VMGIDKHRTFPAYSSQKFETWYKRMAAKKQDSYS |
| | | | KHVSYFHGCYVNYNFPQLGKDLVKIMNAVGYGVH |
| | | | LLEKEKCCGVALIANGLSGQARRQGKVNIRSIRK |
| | | | AAEQNRIVLTTSSTCTFTMRDEYEHLLDIKTDDV |
| | | | RENITLATRFLYRLIEKGDIKLAFRKDFKMRTAY |
| | | | HSACHMEKMGWIIYSTELLKMIPGLELIMLDSQC |
| | | | CGIAGTYGFKKENYQRSQEIGEGLFKQIKELNPD |
| | | | CVSTDCETCKWQIEMSTGYEVKNPISILADALDV |
| | | | EETIKLNQ |
| 19 | Glycerol_uptake_ | P18156 | MMIKNIVLSIPISLIIYLNHLIMEYSMTTQFLME |
| | facilitator_protein | | LIGTLILVLFGDGVCACVTLNKSKGQKAGWVVIT |
| | | | IAWGLAVCMGVLVAGPYTGAHLNPAVSIGLAVAG |
| | | | MFPWSSVPYYIVAQMIGGFLGGLLVWFFYKDHYD |
| | | | ATDDEAAKLGTFCTSPAIRNYKMNFLSEVIATLV |
| | | | LVFIIISFSVDGNTGDAEHFKFGLAALGPIPVTL |
| | | | LIIALGMSLGGTTGYAMNPARDLSPRLAHAVCMK |
| | | | GDNDWSYSWIPVLGPIIGAIIAGFCGAALLLV |
| 20 | Serine/threonine- | Q97PA9 | MSEKIIPSNEPAQAASEPIKASYTEYTVIPSQGY |
| | protein_kinase_StkP | | CQFVKCKKGDQPVVLKGLKEAYRERVLLRNALKR |
| | | | EFKQCQRLNHPGIVRYQGLVDVEGYGLCIEEEYV |
| | | | DGRTLQAYLKESHTDDEKITIVNQIADALRYAHQ |
| | | | QGVAHRNLKPSNILITKQGDHVKLIDFNVLSLDD |
| | | | VKPTADTTRFMAPELKDETMTADGTADIYSLGTI |
| | | | MKVMGLTLAYSEVIKRCCAFKRSDRYSDIDEFLA |
| | | | DFNHDGSSFSMPKIGKGTVVIGFIAVVVIALAAL |
| | | | AYNYGGALVDQVGKIDVTSIFKSDAETAPEDSAM |
| | | | VKSVEQNNNDSVADEAPATGKLAFMNTMKPALYK |
| | | | DLDRLFAKHSDDRAKLNRAIKVYYRGLIQANDTL |
| | | | DNEQRAELDRVFGNYVKQKKAALK |
| 21 | Cluster: D-alanyl- | G6AHI1 | MLVAQLFVGVLQAQKPVQNRRQAVGQSMERQGLV |
| | D-alanine dipeptidase | | NVKAVVPSIKVALMYARTDNFCHRMALS |
| 22 | Anaerobic_C4- | P0ABN5 | MITGLVIIQLLIVLALIFIGARVGGIGLGIYGMI |
| | dicarboxylate_ | | GVFILVYGFGLAPGSAPIDVMMIIVAVITAASAL |
| | transporter_DcuA | | QASGGLEYLVGVAAKFLQKHPDHITYFGPITCWL |
| | | | FCVVAGTAHTSYSLMPIIAEIAQTNKIRPERPLS |
| | | | LSVIAASLGITCSPVSAATAALISQDLLGAKGIE |
| | | | LGTVLMICIPTAFISILVAAFVENHIGKELEDDP |
| | | | EYKRRVAAGLINPEAACEEVQKAENEHDPSAKHA |
| | | | VWAFLFGVALVILFGFLPQLRPEGVSMSQTIEMI |
| | | | MMSDAALILLVGKGKVGDAVNGNIFKAGMNAVVA |
| | | | IFGIAWMGNTFYVGNEKILDAALSSMISSTPILF |
| | | | AVALFLLSIMLFSQAATVTTLYPVGIALGINPLL |
| | | | LIAMFPACNGYFFLPNYPTEVAAIDFDRTGTTRV |
| | | | GKYVINHSFQIPGFITTIVSILLGVLMVQFFR |
| 23 | L-asparaginase_2 | P00805 | MRILKITFVTVLALVMSTVVFAQKPKIRIIATGG |
| | | | TIAGVSASATSSAYGAGQVGVQTLIDAVPQIKDI |
| | | | ADVSGEQLVNIGSQDMNDEVWLKLAKRINDLLNK |
| | | | EGYDGVLITHGTDTMEETAYFLSLTVHTDKPVVM |
| | | | VGSMRPSTAISADGPANLYNGICTLVDPSSKGHG |
| | | | VMVCMNNELFEAKSVIKTHTTDVSTFKGGLYGEM |
| | | | GYVYNGKPYFLHKPVAKQGLTSEFNVDNLTSLPK |
| | | | VGIVYGYANCSPLPIQAFVNAKFDGIVLAGVGDG |
| | | | NFYKDVFDVALKAQNSGIQIVRSSRVPFGPTNLN |
| | | | GEVDDAKYHFVASLNLNPQKARVLLMLALTKTKD |
| | | | WQKIQQYFNEY |
| 24 | Trehalose_synthase/ | P9WQ19 | MALACAMTMSASAQMGTNPKWLGDAIFYQIYPSS |
| | amylase_TreS | | YMDTDGNGIGDLPGITQKLDYIKSLGVNAIWLNP |
| | | | VFESGWFDGGYDVIDFYKIDPRFGTNTDMVNLVK |
| | | | EAHKRGIKVCLDLVAGHTSTKCPWFKESANGDRN |
| | | | SRYSDYFIWTDSISEADKKEIAERHKEANPASST |
| | | | HGRYVEMNAKRGKYYEKNFFECQPALNYGFAKPD |
| | | | PNQPWEQPVTAPGPQAVRREMRNIMAFWFDKGVD |
| | | | GFRVDMASSLVKNDWGKKEVSKLWNEMREWKDKN |
| | | | YPECVLISEWSDPAVAIPAGFNIDFMIHFGIKGY |
| | | | PSLFFDRNTPWGKPWPGQDISKDYKFCYFDKAGK |
| | | | GEVKEFVDNFSEAYNATKNLGYIAIPSANHDYQR |
| | | | PNIGTRNTPEQLKVAMTFFLTMPGVPFIYYGDEI |
| | | | GMKYQMDLPSKEGSNERAGTRTPMQWTSGPTAGF |
| | | | STCNPSQLYFPVDTEKGKLTVEAQQNDPRSLLNY |
| | | | TRELTRLRHSQPALRGNGEWILVSKESQPYPMVY |
| | | | KRTSGGETVVVAINPSDKKVSANIAHLGKAKSLI |
| | | | MTGKASYKTGKTEDAVELNGVSAAVFKIAE |
| 25 | Ribitol-5- | Q720Y7 | MNIAVIFAGGSGLRMHTKSRPKQFLDLNGKPIII |
| | phosphate_cytidyl- | | YTLELFDNHPGIDAIVVACIESWIPFLEKQLRKF |
| | yltransferase | | EINKVVKIVPGGESGQASIYNGLCAAEAYIKSKN |
| | | | VASEDTTVLIHDGVRPLITEETITDNINKVAEVG |
| | | | SCITCIPATETLVVKQHDGSLEIPSRADSLIARA |
| | | | PQSFLLSDILTAHRRAIDEKKNDFIDSCTMMSHY |
| | | | GYRLGTIIGPMENIKITTPTDFFVLRAMVKVHED |
| | | | QQIFGL |
| 26 | UDP-Glc: alpha-D- | B5L3F2 | MTEKKSVSIVLCTYNGTKYLQEQLDSILAQTYPL |
| | GlcNAc- | | HEIIIQDDGSTDNTWQILEKYEEKYPLIHIYHNE |
| | diphosphoundecaprenol | | GTHGVNANFLSAMHRTTGDFIAIADQDDIWETDK |
| | | | IANQMTTIGNKLLCSGLTRPFSSDGSFAYFDNRP |
| | | | RNVSIFRMMFLGLPGHTMLFRRELLRMMPPVTHS |
| | | | FFNVSLYDAALSILAASHDSIAFCNKVLVNFRRH |
| | | | ADATTYNDYSRSLPSWQNGLYELLWGLRHYHQAR |
| | | | SIALPIYRGKLALMEGITTNYHDFIEAKAIMRLE |
| | | | TQKGLWAFLRLQYLLTKNHQRLFQTSGGSFIKMI |
| | | | RAWLYPVMQLYMYHHALRRCK |
| 27 | UDP-N- | P33038 | MESFIIEGGHRLSGTIAPQGAKNEALEVICATLL |
| | acetylglucosamine | | TTEEVIIRNIPNILDVNNLIKLLQDIGVKVKKLG |
| | | | ANDFSFQADEVKLDYLESIDFVKKCSSLRGSVLM |
| | | | IGPLLGRFGKATIAKPGGDKIGRRRLDTHFLGFK |
| | | | NLGARFVRIEDRDVYEIQADKLVGDYMLLDEASV |
| | | | TGTANIIMSAVMAEGTTTIYNAACEPYIQQLCHL |
| | | | LNAMGAKITGIASNLITIEGVTSLHGAEHRILPD |
| | | | MIEVGSFIGMAAMVGDGVRIKDVSIPNLGLILDT |
| | | | FRRLGVQIIEDEDDLIIPRQDHYVIDSFIDGTIM |
| | | | TISDAPWPGLTPDLISVLLVVATQAQGSVLFHQK |
| | | | MFESRLFFVDKLIDMGAQIILCDPHRAVVVGHDH |
| | | | AKKLRAGRMSSPDIRAGIALLIAALTAEGTSRID |
| | | | NIAQIDRGYENIEGRLNALGAKVQRVEIC |
| 28 | Sensor_protein_EvgS | P30855 | MERSGNFYKAIRLGYILISILIGCMAYNSLYEWQ |
| | | | EIEALELGNKKIDELRKEINNINIQMIKFSLLGE |
| | | | TILEWNDKDIEHYHARRMAMDSMLCRFKATYPAE |
| | | | RIDSVRHLLEDKERQMCQIVQILEQQQAINDKIT |
| | | | SQVPVIVQKSVQEQPKKSKRKGFLGIFGKKEEAK |
| | | | PTVTTTMHRSFNRNMRTEQQAQSRRLSVHADSLA |
| | | | ARNAELNRQLQGLVVQIDGKVQTDLQKREAEITA |
| | | | MRERSFIQIGGLTGFVILLLVISYIIIHRNANRI |
| | | | KRYKQETADLIERLQQMAKRNEALITSRKKAVHT |
| | | | ITHELRTPLTAITGYAGLIQKNFNADKTGMYIRN |
| | | | IQQSSDRMREMLNTLLSFFRLDDGKEQPNFSTCR |
| | | | ISSIAHTLESEFMPIAINKGLALTVTNHTDAVVL |
| | | | TDKERILQIGNNLLSNAIKFTENGAVSLTMGYDN |
| | | | GMLKLIVKDTGSGMTEEEQQRVFGAFERLSNAAA |
| | | | KDGFGLGLSIVQRIVTMLGGTIQLKSEKGKGSRF |
| | | | TVEIPMQSAEELPERINKTQIHHNRTLHDIVAID |
| | | | NDKVLLLMLKEMYAQEGIHCDTCTNAAELMEMIR |
| | | | RKEYSLLLTDLNMPDINGFELLELLRTSNVGNSR |
| | | | IIPIIVTTASGSCNREELLERGFSDCLLKPFSIS |
| | | | ELMEVSDKCAMKGKQNEKPDFSSLLSYGNESVML |
| | | | DKLIAETEKEMQSVRDGEQRKDFQELDALTHHLR |
| | | | SSWEILRADQPLRELYKQLHGSAVPDYEALNNAV |
| | | | TAVLDKGSEIIRLAKEERRKYENG |
| 29 | Phosphate- | Q7A5Q2 | MKRSRFYITVGLILSLTLLMSACGQKKAKDGRTD |
| | binding_protein_PstS | | TPTSGTIKFASDESFSPIVEELLQNYQFRYPQAH |
| | | | LLPIYTDDNTGMKLLLDQKVNLFITSHAMTKGED |
| | | | AILRGKGPIPEVFPIGYDGIAFIVNRSNPDSCIT |
| | | | VDDVKKILQGKIAKWNQLNPKNNRGSIEVVFDNK |
| | | | ASATLHYVVDSILGGKNIKSENIVAAKNSKSVID |
| | | | YVNKTPNAIGVIGSNWLNDHRDTTNTTFKKDVTV |
| | | | ASISKATVASPSNSWQPYQAYLLDGRYPFVRTIY |
| | | | ALLADPHKALPYAFANYIANPIGQMIIFKAGLLP |
| | | | YRGNINIREVEVKNQ |
| 30 | Bifunctional_purine_ | P9WHM7 | MAGTKRIKTALISVFHKDGLDDLLKKLDEEGVQF |
| | biosynthesis_protein_ | | LSTGGTQQFIESLGYECQKVEDVTSYPSILGGRV |
| | PurH | | KTLHPKIFGGILARRDNEEDQKQMVEYTIPAIDL |
| | | | VIVDLYPFEQTVASGASAQDIIEKIDIGGISLIR |
| | | | AGAKNFKDVVIVPSKAEYPVLLQLLNTKGAETEI |
| | | | EDRKMFAERAFGVSSHYDTAIHSWFAAE |
| 31 | Multidrug_efflux_ | P0AE06 | MEEEKGGRIGQRPYILKIITERNYIIIIDMKKAK |
| | pump_subunit_AcrA | | ILLFVTALVAVLTSCGGGQKGLPTSDEYPVITIG |
| | | | ASNAQLKTTYPATIKGVQDVEVRPKVSGFITKLN |
| | | | IHEGEYVHAGQVLFVIDNSTYQAAVRQAQAQVNS |
| | | | AQSAVAQAKANVVQANASLNSANAQAATSRLTYN |
| | | | NSQNLYNNKVIGDYELQSAKNTYETAQASVRQAQ |
| | | | SGIASAQAAVKQAEAGVRQAQAMLSTAKDNLGFC |
| | | | YVKSPASGYVGSLPFKEDALVSASSAQPVTTISN |
| | | | TSTIEVYFSMTEADVLKLSRTDDGLSNAIKKFPA |
| | | | VSLLLADGSTYNHEGAIVKTSGMIDATTGTINVI |
| | | | ARFPNPEHLLKSGGSGKIVIAKNNNRALLIPQEA |
| | | | VTQVQNKMFVYKVDAKDKVHYSEITVDPQNDGIN |
| | | | YIVTSGLKMGERIVSKGVSSLEDGAKIKALTPAE |
| | | | YEEAIKKAEKLGENQSSASGFLKTMKGDSK |
| 32 | Cell_division_protein_ | Q81X30 | MAKRRNKARSHHSLQVVTLCISTAMVLILIGMVV |
| | FtsX | | LTVFTSRNLSSYVKENLTVTMILQPDMSTEESAA |
| | | | LCQRIRSLHYINSLNFISKEQALKEGTRELGANP |
| | | | AEFAGQNPFTGEIELQLKANYANNDSIKNIEREL |
| | | | RTYRGVSDITYPQNLVESVNHTLGKISLVLLVIA |
| | | | ILLTIVSFSLMNNTIRLSIYARRFSIHTMKLVGA |
| | | | SWGFIRAPFLRRAVMEGLVSALLAIAVLGVGLCL |
| | | | LYDYEPDITKVLSWDVLVITAGVMLAFGVLIATF |
| | | | CSWLSVNKFLRMKAGDLYKI |
| 33 | Fe(2+)_transporter_ | Q9PMQ9 | MKLSDLKTGETGVIVKVLGHGGFRKRIIEMGFIQ |
| | FeoB | | GKQVEVLLNAPLRDPVKYKIMGYEVSLRHSEADQ |
| | | | IEVISAEEARQLEQAKADNEPQQGALSNNIPDES |
| | | | DHALTPFELTDAANRKSKVINVALVGNPNCGKTS |
| | | | LFNFASGAHERVGNYSGVTVDAKVGRANYEGYEF |
| | | | HLVDLPGTYSLSAYSPEELYVRKQLVEKTPDVVI |
| | | | NVIDASNLERNLYLTTQLIDMHVRMVCALNMFDE |
| | | | TEQRGDNIDYQKISELFGIPMVPTVFTNGRGVKE |
| | | | LFHQVIAVYEGKEDETSQFRHIHINHGHELEGGI |
| | | | KNIQEHLRAYPDICQRYSTRYLAIKLLEHDKDVE |
| | | | ELIKPLKDSDEIFKHRDIAAQRVKEETGNESETA |
| | | | IMDAKYGFIHGALEEADYSTGQKKDTYQTTHFID |
| | | | QILTNKYFGFPIFFLILFIMFTATFVIGQYPMDW |
| | | | IDGGVSWLGDFISSNMPDGPVKDMLVDGIIGGVG |
| | | | AVIVFLPQILILYFFISYMEDSGYMARAAFIMDK |
| | | | LMHKMGLHGKSFIPLIMGFGCNVPAVMATRTIES |
| | | | RRSRLVTMLILPLMSCSARLPIYVMITGSFFALK |
| | | | YRSLAMLSLYVIGILMSVIMSRVFSRFLVKGEDT |
| | | | PFVMELPPYRFPTWKAIGRHTWEKGKQYLKKMGG |
| | | | IILVASIIVWALGYFPLPDKPDMGQQERQEHSFI |
| | | | GQIGHAVEPVFRPQGFNWKLDVGLLAGVGAKEIV |
| | | | ASTMGVLYSNDDSFKDDNSFSSEGGKYVKLHKQI |
| | | | TQDVANLHGVSYNEAEPIATLTAFCFLLFVLLYF |
| | | | PCIATIAAIKGETGSWGWALFAAGYTTLLAWVVS |
| | | | AIVFQVGMLFIG |
| 34 | Pneumolysin | Q04IN8 | MKKNLLKAVLPASLALFAVTFGSCSQDGQLTGTK |
| | | | EDTGERVLDNTREIQNYLRTLPLAPMMSRASDPV |
| | | | PSDDGTTVPVDEGTSKTEEKGVLNGIPGSWVKTT |
| | | | RRYKMTQAFDESFLFDPTSDIVYPGCVLKGGTIA |
| | | | NGTYAIITSHETGDVTFSINLSPANPQEARETSA |
| | | | TVHNIRKSEYQEVWNKWANMQWKESPITTIESVE |
| | | | KINSQEELATKLGVAVNSPVANGSLNFGFNFNKK |
| | | | KNHILARLIQKYFSVSTDAPKKGNIFESIDKEAL |
| | | | DGYQPVYISNINYGRIIYLSVESDEDEKVVDEAI |
| | | | NFAMNQIKGVDVSVSADQSLHYRKVLANCDIRIT |
| | | | VLGGGQTIQKEVLKGDIDSFQRFLNADIPMEQMS |
| | | | PISFSLRYAVDNSQARVVTSNEFTVTQRDFVPEF |
| | | | KKVRMQLQVLGFSGTNTGPFPNLDREAGLWGSIS |
| | | | LSLNGQDNELVKISQSNPFFFNYREKKETMHPIG |
| | | | FGGIVTVEFDKDPNESLEDFVDHQKMTFVSDLHS |
| | | | TRSIYNYNFGRTTFTHTLGTLYTKYKGDDPIFVL |
| | | | ESNNKNVKIHTYVKVLDMKFFN |
| 35 | Cluster: | G6AG77 | MTKFIYAMSLFLLAAISIKAQPIQKTSGCLLHGS |
| | Uncharacterized | | VVSSTDATAIAGATVRLYQLKKLVGGTVSDASGN |
| | protein | | FDVKCPSSGSLQLRITAVGFKEVDTTLNVPTVTP |
| | | | LSIYMRAGKHAMDEVTVTASEKRGMTSTTVIGQT |
| | | | AMEHLQPSSFADLLALLPGGMTKIPALGSANVIT |
| | | | LREAGPPSSQYATSSLGTKFVIDGQAIGTDANMQ |
| | | | YIAGSFQGDADNSRNHVSYGVDMREIPTDNIEKV |
| | | | EVVRGIPSVKYGELTSGLINITRKRSQSPLLLRL |
| | | | KADEYGKLVSVGKGFLLSGKWNLNVDGGLLDARK |
| | | | EPRNRFETYRRLTFSARLRRKWNLGERYVLEWSG |
| | | | ATDYSLNIDNVKTDPEIQIHREDSYRSSYLKMGM |
| | | | NHRLLLRRKALVGLQSVSLAYSASLASDRIHQTE |
| | | | AVALQRDYVVPLAYEGGEYDGLFLPMQYLCDYRV |
| | | | EGKPFYSTLRGETEWLARTSFISHHITAGGEFLL |
| | | | NKNYGRGQIFDITKPLHASTARRPRSYKDIPATD |
| | | | ILSFYAEDKATMPIGKHQLTVMAGLRTTQMLNIP |
| | | | ASYAVHGKLFTDTRVNVQWDFPSFLGFKSFVSGG |
| | | | LGMMTKMPTVLDLYPDYVYKDITEMNYWDIRPAY |
| | | | KRIHIRTYKLNQVNPDLRPARNKKWEIRLGMDKG |
| | | | AHHFSVTYFHEDMKDGFRSTTTMRPFIYKRYDTS |
| | | | VINPSALTGPPSLASLPVVTDTLLDGYGRTENGS |
| | | | RITKQGIEFQYSSPRIPVIQTRITVNGAWFRTLY |
| | | | ENSIPLFRSAPNVVVGTVAIADRYAGYYMSTDKY |
| | | | DKQIFTSNFIFDSYVDKLGLILSATAECFWMSNT |
| | | | KRPATSSTPMGYMDITGTVHPYVEADQSDPYLRW |
| | | | LVLTGTAGQDMDYRERSYMLVNFKATKRFGRHLS |
| | | | LSFFADRVFYVAPDYEVNGFIVRRTFSPYFGMEI |
| | | | GLKI |
| 36 | Cell_division_ATP- | P0A9R7 | MLIDFKKVNIYQDERLILKDIDFQATEGEFIYLI |
| | binding_protein_FtsE | | GRVGSGKSSLLKTFYGELDIDQEDAEKAEVLGES |
| | | | VLDIKQKRIPALRRQMGIIFQDFQLLHDRSVAKN |
| | | | LKFVLQATGWKDKEKIKQRIKEVLEQVGMIDKAA |
| | | | KMPSELSGGEQQRIAIARAFLNNPKIILADEPTG |
| | | | NLDPETASNIVSILKDTCKNGTTVIMSTHNINLL |
| | | | SQFPGKVYRCMEQALVPVTNEAQTKDLEEDSTSV |
| | | | EPLIEPVLEEEAQAEDSKE |
| 37 | Di-/tripeptide_ | P0C2U3 | MFENQPKALYALALANTGERFGYYTMIAVFALFL |
| | transporter | | RANFGLEPGTAGLIYSIFLGLVYFLPLIGGIMAD |
| | | | KFGYGKMVTIGIIVMFAGYLFLSVPLGGGTVAFG |
| | | | AMLAALLLISFGTGLFKGNLQVMVGNLYDTPELA |
| | | | SKRDSAFSIFYMAINIGALFAPTAAVKIKEWAET |
| | | | SLGYAGNDAYHFSFAVACVSLIVSMGIYYAFRST |
| | | | FKHVEGGTKKTEKAAAAAVEELTPQQTKERIVAL |
| | | | CLVFAVVIFFWMAFHQNGLTLTYFADEFVSPTST |
| | | | GVQSMAFDVVNLVMIVFIVYSIMALFQSKTTKAK |
| | | | GIACAVILAAIAVLAYKYMNVNGQVEVSAPIFQQ |
| | | | FNPFYVVALTPISMAIFGSLAAKGKEPSAPRKIA |
| | | | YGMIVAGCAYLLMVLASQGLLTPHEQKLAKAAGE |
| | | | TVPFASANWLIGTYLVLTFGELLLSPMGISFVSK |
| | | | VAPPKYKGAMMGGWFVATAIGNILVSVGGYLWGD |
| | | | LSLTVVWTVFIVLCLVSASFMFLMMKRLEKVA |
| 38 | Calcium- | Q47910 | MKKILIFVAGLCMSLAASAQIQRPKLVVGLVVDQ |
| | transporting_ATPase | | MRWDYLYYYYNEYGTDGLRRLVDNGFSFENTHIN |
| | | | YAPTVTAIGHSSVYTGSVPAITGIAGNYFFQDDK |
| | | | NVYCCEDPNVKSVGSDSKEGQMSPHRLLASTIGD |
| | | | ELQISNDFRSKVIGVALKDRASILPAGHAADAAY |
| | | | WWDTSAGHFVTSTFYTDHLPQWVIDFNEKNHTAP |
| | | | NFNIKTSTQGVTMTFKMAEAALKNENLGKGKETD |
| | | | MLAVSISSTDAIGHVYSTRGKENHDVYMQLDKDL |
| | | | AHFLKTLDEQVGKGNYLLFLTADHGAAHNYNYMK |
| | | | EHRIPAGGWDYRQSVKDLNGYLQGKFGIAPVMAE |
| | | | DDYQFFLNDSLIAASGLKKQQIIDESVEYLKKDP |
| | | | RYLYVFDEERISEVTMPQWIKERMINGYFRGRSG |
| | | | EIGVVTRPQVFGAKDSPTYKGTQHGQPFPYDTHI |
| | | | PFLLYGWNVKHGATTQQTYIVDIAPTVCAMLHIQ |
| | | | MPNGCIGTARNMALGN |
| 39 | Poly-beta-1,6-N- | Q5HKQ0 | MDRQVFQTDSRQRWNRFKWTLRVLITIAILLGVV |
| | acetyl-D- | | FVAMFALEGSPQMPFRHDYRSVVSASEPLLKDNK |
| | glucosamine_synthase | | RAEVYKSFRDFFKEQKMHSNYAKVAARQHRFVGH |
| | | | TDNVTQKYIKEWTDPRMGIRSAWYVNWDKHAYIS |
| | | | LKNNLKNLNMVLPEWYFINPKTDRIEARIDQRAL |
| | | | KLMRRAHIPVLPMLTNNYNSAFRPEAIGRIMRDS |
| | | | TKRMGMINELVAACKHNGFAGINLDLEELNINDN |
| | | | ALLVTLVKDFARVFHANGLYVTQAVAPFNEDYDM |
| | | | QELAKYDDYLFLMAYDEYNAGSQAGPVSSQRWVE |
| | | | KATDWAAKNVPNDKIVLGMATYGYNWAQGQGGTT |
| | | | MSFDQTMATALNAGAKVNFNDDTYNLNFSYQDED |
| | | | DGTLHQVFFPDAVTTFNIMRFGATYHLAGFGLWR |
| | | | LGTEDSRIWKYYGKDLSWESAARMPIAKIMQLSG |
| | | | TDDVNFVGSGEVLNVTSEPHAGRIGIVLDKDNQL |
| | | | IIEERYLSLPATYTVQRLGKCKEKQLVLTFDDGP |
| | | | DSRWTPKVLSILKHYKVPAAFFMVGLQIEKNIPI |
| | | | VKDVFNQGCTIGNHTFTHHNMIENSDRRSFAELK |
| | | | LTRMLIESITGQSTILFRAPYNADADPTDHEEIW |
| | | | PMIIASRRNYLFVGESIDPNDWQQGVTADQIYKR |
| | | | VLDGVHQEYGHIILLHDAGGDTREPTVTALPRII |
| | | | ETLQREGYQFISLEKYLGMSRQTLMPPIKKGKEY |
| | | | YAMQANLSLAELIYHISDFLTALFLVFLVLGFMR |
| | | | LVFMYVLMIREKRAENRRNYAPIDPLTAPAVSII |
| | | | VPAYNEEVNIVRTISNLKEQDYPSLKIYLVDDGS |
| | | | KDNTLQRVREVFENDDKVVIISKKNGGKASALNY |
| | | | GIAACSTDYIVCVDADTQLYKDAVSKLMKHFIAD |
| | | | KTGKLGAVAGNVKVGNQRNMLTYWQAIEYTTSQN |
| | | | FDRMAYSNINAITVIPGAIGAFRKDVLEAVGGFT |
| | | | TDTLAEDCDLTMSINEHGYLIENENYAVAMTEAP |
| | | | ESLRQFIKQRIRWCFGVMQTFWKHRASLFAPSKG |
| | | | GFGMWAMPNMLIFQYIIPTFSPIADVLMLFGLFS |
| | | | GNASQIFIYYLIFLLVDASVSIMAYIFEHESLWV |
| | | | LLWIIPQRFFYRWIMYYVLFKSYLKAIKGELQTW |
| | | | GVLKRTGHVKGAQTIS |
| 40 | ATP_synthase_subunit_ | P29707 | MSQINGRISQIIGPVIDVYFDTKGENPEKVLPNI |
| | beta,_sodium_ion_ | | YDALRVKKADGQDLIIEVQQQIGEDTVRCVAMDN |
| | specific | | TDGLQRGLEVVPTGSPIVMPAGEQIKGRMMNVIG |
| | | | QPIDGMSALQMEGAYPIHREAPKFEDLSTHKEML |
| | | | QTGIKVIDLLEPYMKGGKIGLFGGAGVGKTVLIM |
| | | | ELINNIAKGHNGYSVFAGVGERTREGNDLIRDML |
| | | | ESGVIRYGEKFRKAMDEGKWDLSLVDSEELQKSQ |
| | | | ATLVYGQMNEPPGARASVALSGLTVAEEFRDHGG |
| | | | KNGEAADIMFFIDNIFRFTQAGSEVSALLGRMPS |
| | | | AVGYQPTLASEMGAMQERITSTKHGSITSVQAVY |
| | | | VPADDLTDPAPATTFTHLDATTELSRKITELGIY |
| | | | PAVDPLGSTSRILDPLIVGKEHYDCAQRVKQLLQ |
| | | | KYNELQDIIAILGMDELSDDDKLVVNRARRVQRF |
| | | | LSQPFTVAEQFTGVKGVMVPIEETIKGFNAILNG |
| | | | EVDDLPEQAFLNVGTIEDVKEKAKQLLEATKA |
| 41 | Cluster: | G6AGX5 | MNPIYKIITSILFCVLSINTMAQDLTGHVTSKAD |
| | Uncharacterized | | DKPIAYATVTLKENRLYAFTDEKGNYTIKNVPKG |
| | protein | | KYTVVFSCMGYASQTVVVMVNAGGATQNVRLAED |
| | | | NLQLDEVQVVAHRKKDEITTSYTIDRKTLDNQQI |
| | | | MTLSDIAQLLPGGKSVNPSLMNDSKLTLRSGTLE |
| | | | RGNASFGTAVEVDGIRLSNNAAMGETAGVSTRSV |
| | | | SASNIESVEVVPGIASVEYGDLTNGVVKVKTRRG |
| | | | SSPFIVEGSINQHTRQIALHKGVDLGGNVGLLNF |
| | | | SIEHARSFLDAASPYTAYQRNVLSLRYMNVFMKK |
| | | | SLPLTLEVGLNGSIGGYNSKADPDRSLDDYNKVK |
| | | | DNNVGGNIHLGWLLNKRWITNVDLTAAFTYADRL |
| | | | SESYTNESSNATQPYIHTLTEGYNIAEDYDRNPS |
| | | | ANIILGPTGYWYLRGFNDSKPLNYSLKMKANWSK |
| | | | AFGKFRNRLLVGGEWTSSMNRGRGTYYADMRYAP |
| | | | SWREYRYDALPSLNNIAIYAEDKLSMDVNERQNA |
| | | | ELTAGIREDITSIPGSEYGSVGSFSPRMNARYVF |
| | | | RFGQNSWLNSMTLHAGWGRSVKIPSFQVLYPSPS |
| | | | YRDMLAFASTSDADNRSYYAYYTYPSMARYNANL |
| | | | KWQRADQWDLGVEWRTKIADVSLSFFRSKVSNPY |
| | | | MATDVYTPFTYKYTSPAMLQRSGIAVADRRFSID |
| | | | PQTGIVTVSDASGVKSPVTLGYEERNTYVTNTRY |
| | | | VNADALQRYGLEWIVDFKQIKTLRTQVRLDGKYY |
| | | | HYKAQDETLFADVPVGLNTRQSDGRLYQYVGYYR |
| | | | GGAATTTNYTANASASNGSVSGQVDLNATITTHI |
| | | | PKIRLIVALRLESSLYAFSRATSSRGYVVSSGNE |
| | | | YFGVPYDDKTENQTVIVYPEYYSTWDAPDVLIPF |
| | | | AEKLRWAETNDRGLFNDLAQLVVRTNYPYTLNPN |
| | | | RLSAYWSANLSVTKEIGRHVSVSFYANNFFNTLS |
| | | | QVHSTQTGLETSLFGSGYVPSFYYGLSLRLKI |

In some embodiments, the Prevotella bacteria is a strain of Prevotella bacteria free or substantially free of one or more (e.g., 1, 2, 3, 4, 5, 6, 7, 8, 9, 10, 11, 12, 13, 14, 15, 16, 17, 18, 19, 20, 21, 22, 23, 24, 25 or more) proteins listed in Table 2 and/or one or more (e.g., 1, 2, 3, 4, 5, 6, 7, 8, 9, 10, 11, 12, 13, 14, 15, 16, 17, 18, 19, 20, 21, 22, 23, 24, 25 or more) genes encoding proteins listed in Table 2. In some embodiments, Prevotella bacteria is free of all of the proteins listed in Table 2 and/or all of the genes encoding the proteins listed in Table 2.

TABLE 2
Other Prevotella proteins

| Seq. ID. No. | Name | Uniprot ID | Amino Acid Sequence |
|---|---|---|---|
| 42 | UDP-Gal: alpha-D- | Q03084 | MERIDISVLMAVYKKDNPAFLRESLESIFSQTVEAAEV |
| | GlcNAc- | | VLLEDGPLTDALYDVIKSYEAIYSTLKVVSYPENRGLG |
| | diphosphounde- | | KTLNDGLLLCKYNLVARMDADDICKPNRLEMEYNWLKS |
| | caprenol | | HEDYDVIGSWVDEFTDNKTRVKSIRKVPEAYDEIKNYA |
| | | | QYRCPINHPTAMYRKAAVLAVGGYLTEYFPEDYFLWLR |
| | | | MLNNGSKFYNIQESLLWFRYSEETVAKRGGWAYACDEV |
| | | | RILVRMLKMGYIPFHVFCQSVVIRFTTRVMPLPIRQRL |
| | | | YNLIRKT |
| 43 | ATP_synthase_ | A1B8P0 | MSQINGRISQIIGPVIDVYFDTKGENPEKVLPKIHDAL |
| | subunit_beta | | RVKRANGQDLIIEVQQHIGEDTVRCVAMDNTDGLQRNL |
| | | | EVVPTGSPIVMPAGDQIKGRMMNVIGQPIDGMEALSME |
| | | | GAYPIHREAPKFEDLSTHKEMLQTGIKVIDLLEPYMKG |
| | | | GKIGLFGGAGVGKTVLIMELINNIAKGHNGYSVFAGVG |
| | | | ERTREGNDLIRDMLESGVIRYGEKFRKAMDEGKWDLSL |
| | | | VDQEELQKSQATLVYGQMNEPPGARASVALSGLTVAEE |
| | | | FRDHGGKNGEAADIMFFIDNIFRFTQAGSEVSALLGRM |
| | | | PSAVGYQPTLASEMGTMQERITSTKHGSITSVQAVYVP |
| | | | ADDLTDPAPATTFTHLDATTELSRKITELGIYPAVDPL |
| | | | GSTSRILDPLIVGKDHYECAQRVKQLLQHYNELQDIIA |
| | | | ILGMDELSDEDKLVVNRARRVQRFLSQPFTVAEQFTGV |
| | | | KGVMVPIEETIKGFNAILNGEVDDLPEQAFLNVGTIED |
| | | | VKEKAKRLLEATK |
| 44 | Cell_division_ | O05779 | MPIGNGQKYQLTIINHTEIIMLIDYKKVNIYQDERLIL |
| | ATP-binding_ | | KDVDFQAETGEFIYLIGRVGSGKSSLLKTIYGELDIDS |
| | protein_FtsE | | EDAEKAVVLDESMPNIKRSRIPALRKQMGIIFQDFQLL |
| | | | HDRSVAKNLKFVLQATGWTSKQKIERRIEEVLAQVGMT |
| | | | DKKNKMPSELSGGEQQRIAIARALLNTPKIIIADEPTG |
| | | | NLDPETAANIVSILKDSCQAGTTVIMSTHNINLIDQFP |
| | | | GKVYRCHEGELHQLTDKKEVSELAEETAPVETIDEPEQ |
| | | | ND |
| 45 | Hemin_transport_ | Q56992 | MKRNILLFICLATSILLLFGLNLTTGSVQIPFADILDI |
| | system_permease_ | | LCGRFIGKESWEYIILENRLPQTLTAILCGASLSVCGL |
| | protein_HmuU | | MLQTAFRNPLAGPDVFGISSGAGLGVALVMLLLGGTVS |
| | | | TSIFTVSGFLAILTAAFVGAIAVTALILFLSTLVRNSV |
| | | | LLLIVGIMVGYVSSSAVSLLNFFASEEGVKSYMVWGMG |
| | | | NFGAVSMNHIPLFSILCLIGIIASFLLVKPLNILLLGP |
| | | | QYAESLGISTRQIRNILLVVVGLLTAITTAFCGPISFI |
| | | | GLAIPHIARLLFRTENHQILLPGIVLSGAAIALLCNFI |
| | | | CYLPGESGIIPLNAVTPLIGAPIIIYVIIQRR |
| 46 | Hexuronate_ | O34456 | MKKYYPWVLVALLWFVALLNYMDRQMLSTMQEAMKVDI |
| | transporter | | AELNHAEAFGALMAVFLWIYGIVSPFAGIIADRVNRKW |
| | | | LVVGSIFVWSAVTYLMGYAESFDQLYWLRAFMGISEAL |
| | | | YIPAALSLIADWHEGKSRSLAIGIHMTGLYVGQAVGGF |
| | | | GATLAAMFSWHAAFHWFGIIGIVYSLVLLLFLKENPKH |
| | | | GQKSVLQGETKPSKNPFRGLSIVFSTWAFWVILFYFAV |
| | | | PSLPGWATKNWLPTLFANSLDIPMSSAGPMSTITIAVS |
| | | | SFIGVIMGGVISDRWVQRNLRGRVYTSAIGLGLTVPAL |
| | | | MLLGFGHSLVSVVGAGLCFGIGYGMFDANNMPILCQFI |
| | | | SSKYRSTAYGIMNMTGVFAGAAVTQVLGKWTDGGNLGN |
| | | | GFAILGGIVVLALVLQLSCLKPTTDNME |
| 47 | 1,4-alpha- | P9WN45 | MVTKKTTTKKAPVKKTSAKTTKVKEPSHIGLVKNDAYL |
| | glucan_branching_ | | APYEDAIRGRHEHALWKMNQLTQNGKLTLSDFANGHNY |
| | enzyme_GlgB | | YGLHQTADGWVFREWAPNATEIYLVGDFNGWNEQEAYQ |
| | | | CHRIEGTGNWELTLPHDAMQHGQYYKMRVHWEGGEGER |
| | | | IPAWTQRVVQDEASKIFSAQVWAPAEPYVWEKKTFKPQ |
| | | | TSPLLIYECHIGMAQDEEKVGTYNEFREKVLPRIIKDG |
| | | | YNAIQIMAIQEHPYYGSFGYHVSSFFAASSRFGTPEEL |
| | | | KALIDEAHKNGIAVIMDIVHSHAVKNEVEGLGNLAGDP |
| | | | NQYFYPGERHEHPAWDSLCFDYGKDEVLHFLLSNCKYW |
| | | | LEEYHFDGFRFDGVTSMLYYSHGLGEAFCNYADYFNGH |
| | | | QDDNAICYLTLANCLIHEVNKNAVTIAEEVSGMPGLAA |
| | | | KFKDGGYGFDYRMAMNIPDYWIKTIKELPDEAWKPSSI |
| | | | FWEIKNRRSDEKTISYCESHDQALVGDKTIIFRLVDAD |
| | | | MYWHFRKGDETEMTHRGIALHKMIRLATIAAINGGYLN |
| | | | FMGNEFGHPEWIDFPREGNGWSHKYARRQWNLVDNEEL |
| | | | CYHLLGDFDRKMLEVITSEKKFNETPIQEIWHNDGDQI |
| | | | LAFSRGELVFVFNFSPSHSYSDYGFLVPEGSYNVVLNT |
| | | | DAREFGGFGFADDTVEHFTNSDPLYEKDHKGWLKLYIP |
| | | | ARSAVVLRKK |
| 48 | Cluster: YihY | D9RW24 | MKIDIERIKYFLTVGMFMKTEHSSKRRNMLIRQFQKFY |
| | family protein | | LTVKFFFVRDHAASTAQLSFSTIMAIVPIASMIFAIAN |
| | | | GFGFGQFLEKQFREMLSAQPEAATWLLKLTQSYLVHAK |
| | | | TGLFIGIGLMIMLYSVFSLIRTVETTFDNIWQVKDSRP |
| | | | ISRIVIDYTALMFLVPISIIILSGLSIYFYSFVENLNG |
| | | | LRFLGTIASFSLRYLVPWAILTLMFIVLYVFMPNAKVK |
| | | | ITKTVAPAMIASIAMLCLQAVYIHGQIFLTSYNAIYGS |
| | | | FAALPLFMLWILASWYICLFCAELCYFNQNLEYYECLI |
| | | | DTEDICHNDLLILCATVLSHICQRFANDQKPQTALQIK |
| | | | TETHIPIRVMTDILYRLKEVNLISENFSPTSDEVTYTP |
| | | | THDTNNITVGEMIARLESTPASDFALLGFSPKKAWNHD |
| | | | IYDRVGSIREIYLNELKSINIKELISYSEN |
| 49 | Capsule_ | P19579 | MMKRPSIARWKVIICLLTPILLSFSGIGDNDIDKKKST |
| | biosynthesis_ | | SKEVDDTLRIVITGDLLLDRGVRQKIDMAGVDALFSPT |
| | protein_CapA | | IDSLFHSSNYVIANLECPVTKIRERVFKRFIFRGEPEW |
| | | | LPTLRRHGITHLNLANNHSIDQGRNGLLDTQEQIKKAG |
| | | | MIPIGAGKNMEEAAEPVLISTSPRHVWVISSLRLPLEN |
| | | | FLYLPQKPCVSQESIDSLIMRVKRLRATDKNCYILLIL |
| | | | HWGWEHHFRATPQQREDAHKLIDAGADAIVGHHSHTLQ |
| | | | TIETYRGKPIYYGIGNFIFDQRKPMNSRACLVELSITA |
| | | | EKCKAKALPIEIKNCTPYLSK |
| 50 | Peptidoglycan_ | B5ZA76 | MILLSFDTEEFDVPREHGVDFSLEEGMKVSIEGTNRIL |
| | deacetylase | | DILKANNVCATFFCTGNFAELAPEVMERIKNEGHEVAC |
| | | | HGVDHWQPKPEDVFRSKEIIERVTGVKVAGYRQPRMFP |
| | | | VSDEDIEKAGYLYNSSLNPAFIPGRYMHLTTSRTWFMQ |
| | | | GKVMQIPASVSPHLRIPLFWLSMHNFPEWFYLRLVRQV |
| | | | LRHDGYFVTYFHPWEFYDLKSHPEFKMPFIIKNHSGHE |
| | | | LEQRLDRFIKAMKADKQEFITYVDFVNRQKK |
| 51 | Fumarate_ | P0AC47 | MAKNISFTIKYWKQNGPQDQGHFDTHEMKNIPDDTSFL |
| | reductase_iron- | | EMLDILNEELIAAGDEPFVFDHDCREGICGMCSLYING |
| | sulfur_subunit | | TPHGKTERGATTCQLYMRRFNDGDVITVEPWRSAGFPV |
| | | | IKDCMVDRTAFDKIIQAGGYTTIRTGQAQDANAILISK |
| | | | DNADEAMDCATCIGCGACVAACKNGSAMLFVSSKVSQL |
| | | | ALLPQGKPEAAKRAKAMVAKMDEVGFGNCTNTRACEAV |
| | | | CPKNEKIANIARLNREFIKAKFAD |
| 52 | Serine/threonine- | P9WI71 | MSENKLSTNEQAQTADAPVKASYTEYKVIPSQGYCMIV |
| | protein_kinase_ | | KCRKGDQTVVLKTLKEEYRERVLLRNALKREFKQCQRL |
| | PknH | | NHSGIVRYQGLVEVDGYGLCIEEEYVEGRTLQAYLKEN |
| | | | HTDDEKIAIINQIADALRYAHQQGVIHRNLKPSNVLVT |
| | | | TQGDYVKLIDFSVLSPEDVKPTAETTRFMAPEMKDETL |
| | | | TADATADIYSLGTIMKVMGLTLAYSEVIKRCCAFKRSD |
| | | | RYSNVDELLADLNNEGSSFSMPKIGKGTVVLGLIIAVV |
| | | | IGIGALLYNYGGALIDQVGKIDVSSVFSSDAETAPEDT |
| | | | VKVNTAEQSDSLSTEAEAPAIGKLAFMNRMKPALYKDL |
| | | | DNIFEKNSADKAKLTKAIKTYYRGLIQANDTLDNEQRA |
| | | | EVDRVFGDYVKQKKAALN |
| 53 | Carboxy- | O34666 | MRKYICLLLFYLFTFLPLSAQQGNDSPLRKLQLAEMAI |
| | terminal_ | | KNFYVDSVNEQKLVEDGIRGMLEKLDPHSTYTDAKETK |
| | processing_ | | AMNEPLQGDFEGIGVQFNMIEDTLVVIQPVVNGPSQKV |
| | protease_CtpA | | GILAGDRIVSVNDSTIAGVKMARIDIMKMLRGKKGTKV |
| | | | KLGVVRRGVKGVLTFVVTRAKIPVHTINASYMIRPNVG |
| | | | YIRIESFGMKTHDEFMSAVDSLKKKGMKTLLLDLQDNG |
| | | | GGYLQSAVQISNEFLKNNDMIVYTEGRRARRQNFKAIG |
| | | | NGRLQDVKVYVLVNELSASAAEIVTGAIQDNDRGTVVG |
| | | | RRTFGKGLVQRPFDLPDGSMIRLTIAHYYTPSGRCIQK |
| | | | PYTKGDLKDYEMDIEKRFKHGELTNPDSIQFSDSLKYY |
| | | | TIRKHRVVYGGGGIMPDNFVPLDTTKFTRYHRMLAAKS |
| | | | IIINAYLKYADANRQALKAQYSSFDAFNKGYVVPQSLL |
| | | | DEIVAEGKKEKIEPKDAAELKATLPNIALQIKALTARD |
| | | | IWDMNEYFRVWNTQSDIVNKAVALATGK |
| 54 | Cluster: | D9RRG3 | MKLTEQRSSMLHGVLLITLFACAAFYIGDMGWVKALSL |
| | Uncharacterized | | SPMVVGIILGMLYANSLRNNLPDTWVPGIAFCGKRVLR |
| | protein | | FGIILYGFRLTFQDVVAVGFPAIIVDAIIVSGTILLGV |
| | | | LVGRLLKMDRSIALLTACGSGICGAAAVLGVDGAIRPK |
| | | | PYKTAVAVATVVIFGTLSMFLYPILYRAGIFDLSPDAM |
| | | | GIFAGSTIHEVAHVVGAGNAMGAAVSNSAIIVKMIRVM |
| | | | MLVPVLLVIAFFVAKNVAERDDEAGGSRKINIPWFAIL |
| | | | FLVVIGFNSLNLLPKELVDFINTLDTFLLTMAMSALGA |
| | | | ETSIDKFKKAGFKPFLLAAILWCWLIGGGYCLAKYLVP |
| | | | VLGVAC |
| 55 | Cluster: Cna | X6Q2J4 | MNKQFLLAALWLSPLGLYAHKANGIGAVTWKNEAPKER |
| | protein B-type | | MIRGIDEDKTHQRFTLSGYVKDRNGEPLINATIYDLTT |
| | domain protein | | RQGTMTNAYGHFSLTLGEGQHEIRCSYVGYKTLIETID |
| | | | LSANQNHDIILQNEAQLDEVVVTTDLNSPLLKTQTGKL |
| | | | SLSQKDIKTEYALLSSPDVIKTLQRTSGVADGMELASG |
| | | | LYVHGGNGDENLFLLDGTPLYHTNHSLGLFSSFNADVV |
| | | | KNVDFYKSGFPARYGGRLSSVIDVRTADGDLYKTHGSY |
| | | | RIGLLDGAFHIGGPIRKGKTSYNFGLRRSWMDLLTRPA |
| | | | FAIMNHKSDNEDKLSMSYFFHDLNFKLTNIFNERSRMS |
| | | | LSVYSGEDRLDAKDEWHSNNSSGYNDVDIYVNRFHWGN |
| | | | FNAALDWNYQFSPKLFANFTAVYTHNRSTVSSSDEWRF |
| | | | TRPGEKEQLTLTSHGYRSSIDDIGYRAAFDFRPSPRHH |
| | | | IRFGQDYTYHRFQPQTYNRFDNYQTNSEAKADTIATHS |
| | | | YNKNVAHQLTFYAEDEMTLNEKWSLNGGVNADVFHISG |
| | | | KTFATLSPRLSMKFQPTERLSLKASYTLMSQFVHKIAN |
| | | | SFLDLPTDYWVPTTARLHPMRSWQVAAGAYMKPNKHWL |
| | | | LSLEAYYKRSSHILQYSSWAGLEPPAANWDYMVMEGDG |
| | | | RSYGVELDADYNVSNLTLHGSYTLSWTQKKFDDFYDGW |
| | | | YYDKFDNRHKLTLTGRWNITKKIAAFAAWTFRTGNRMT |
| | | | IPTQYIGLPDVPAQEQGGLTFNSSDDNTLNFAYEKPNN |
| | | | VILPAYHRLDIGFDFHHTTKKGHERIWNLSFYNAYCHL |
| | | | NSLWVRVKIDSNNQMKIRNIAFIPVIPSFSYTFKF |
| 56 | Poly-beta-1,6-N- | P75905 | MSKQVFQTDSRQRWSYFKWTLRVILTILSLLGIVFLAM |
| | acetyl-D- | | FALEGSPQMPFRHDYRNAVTAASPYTKDNKTAKLYKSF |
| | glucosamine_ | | RDFFKEKKMHNNYAKATIKKQRFIGKADSVTQKYFREW |
| | synthase | | DDPRIGVRSAWYVNWDKHAYISLKNNIKHLNMVLPEWF |
| | | | FINPKTDKVEYRIDKQALRLMRRTGIPVLPMLTNNYNS |
| | | | DFHPEAIGRIMRDEKKRMALINEMVRTCRHYGFAGINL |
| | | | DLEELNIQDNDLLVELLKDFSRVFHANGLYVTQAVAPF |
| | | | NEDYNMQELAKYNDYLFLMAYDEHNIESQPGAVSSQRW |
| | | | VEKATDWAAKNVPNDKIVLGMATYGYDWANGEGGTTVS |
| | | | FDQTMAIAQDADAKVKFDDDTYNVNFSYQNTDDGKIHH |
| | | | VFFTDAATTFNIMRFGAEYHLAGYGLWRLGTEDKRIWR |
| | | | FYGKDMSWENVARMSVAKLMQLNGTDDVNFVGSGEVLE |
| | | | VTTEPHPGDISIRIDKDNRLISEEYYRALPSTYTIQRL |
| | | | GKCKDKQLVITFDDGPDSRWTPTVLSTLKKYNVPAAFF |
| | | | MVGLQMEKNLPLVKQVYEDGHTIGNHTFTHHNMIENSD |
| | | | RRSYAELKLTRMLIESVTGHSTILFRAPYNADADPTEH |
| | | | EEIWPMIVASRRNYLFVGESIDPNDWEPNVTSDQIYQR |
| | | | VIDGVHHEDGHIILLHDAGGSSRKPTLDALPRIIETLQ |
| | | | HEGYQFISLEQYLGMGKQTLMPEINKGKAYYAMQTNLW |
| | | | LAEMIYHVSDFLTALFLVFLALGMMRLIFMYVLMIREK |
| | | | RAENRRNYAPIDAATAPAVSIIVPGYNEEVNIVRTITT |
| | | | LKQQDYPNLHIYFVDDGSKDHTLERVHEAFDNDDTVTI |
| | | | LAKKNGGKASALNYGIAACRSEYVVCIDADTQLKNDAV |
| | | | SRLMKHFIADTEKRVGAVAGNVKVGNQRNMLTYWQAIE |
| | | | YTSSQNFDRMAYSNINAITVVPGAIGAFRKEVIEAVGG |
| | | | FTTDTLAEDCDLTMSINEHGYIIENENYAVALTEAPET |
| | | | LRQFVKQRIRWCFGVMQAFWKHRSSLFAPSKKGFGLWA |
| | | | MPNMLIFQYIIPTFSPLADVLMLIGLFTGNALQIFFYY |
| | | | LIFLVIDASVSIMAYIFEGERLWVLLWVIPQRFFYRWI |
| | | | MYYVLFKSYLKAIKGELQTWGVLKRTGHVKG |
| 57 | Cell_division_ | O34876 | MAKKRNKARSRHSLQVVTLCISTAMVLMLIGIVVLTGF |
| | protein_FtsX | | TSRNLSSYVKENLTITMILQPDMNTEESAALCERIRTL |
| | | | HYINSLNFISKEQALKDGTKELGANPAEFAGENPFTGE |
| | | | IEVQLKANYANNDSIRNIVQQLRTYRGVSDITYPQSLV |
| | | | ESVNQTLGKISLVLLVIAVLLTIISFSLINNTIRLSIY |
| | | | AHRFSIHTMKLVGGSWSFIRAPFLRRAVLEGLVSALLA |
| | | | IAVLGIGICLLYEKEPEITKLLSWDALIITAIVMLAFG |
| | | | VIIATFCAWLSVNKFLRMKAGDLYKI |
| 58 | UDP-2,3- | P44046 | MKNIYFLSDAHLGSLAIDHRRTHERRLVRFLDSIKHKA |
| | diacylglucosamine_ | | AAVYLLGDMFDFWNEYKYVVPKGFTRFLGKISELTDMG |
| | hydrolase | | VEVHFFTGNHDLWTYGYLEKECGVILHRKPITTEIYDK |
| | | | VFYLAHGDGLGDPDPMFRFLRKVFHNRFCQRLLNFFHP |
| | | | WWGMQLGLNWAKRSRLKRKDGKEVPYLGEDKEYLVQYT |
| | | | KEYMSTHKDIDYYIYGHRHIELDLTLSRKARLLILGDW |
| | | | IWQFTYAVFDGEHMFLEEYVEGESKP |
| 59 | Poly-beta-1,6-N- | P75905 | MVGLDVLCYFIHAKGREKECYFERIIYQITCHSRTKCY |
| | acetyl-D- | | LCNIMKYSIIVPVFNRPDEVEELLESLLSQEEKDFEVV |
| | glucosamine_ | | IVEDGSQIPCKEVCDKYADKLDLHYYSKENSGPGQSRN |
| | synthase | | YGAERAKGEYLLILDSDVVLPKGYICAVSEELKREPAD |
| | | | AFGGPDCAHESFTDTQKAISYSMTSFFTTGGIRGGKKK |
| | | | LDKFYPRSFNMGIRRDVYQELGGFSKMRFGEDIDFSIR |
| | | | IFKAGKRCRLFPEAWVWHKRRTDFRKFWKQVYNSGIAR |
| | | | INLYKKYPESLKLVHLLPMVFTVGTALLVLMILFGLFL |
| | | | QLFPIINVFGSVFIMMGLMPLVLYSVIICVDSTMQNNS |
| | | | LNIGLLSIEAAFIQLTGYGCGFISAWWKRCVCGMDEFA |
| | | | AYEKNFYK |
| 60 | Enolase | Q8DTS9 | MKIEKVHAREIMDSRGNPTVEVEVTLENGVMGRASVPS |
| | | | GASTGENEALELRDGDKNRFLGKGVLKAVENVNNLIAP |
| | | | ALKGDCVLNQRAIDYKMLELDGTPTKSKLGANAILGVS |
| | | | LAVAQAAAKALNIPLYRYIGGANTYVLPVPMMNIINGG |
| | | | AHSDAPIAFQEFMIRPVGAPSEKEGIRMGAEVFHALAK |
| | | | LLKKRGLSTAVGDEGGFAPKFDGIEDALDSIIQAIKDA |
| | | | GYEPGKDVKIAMDCAASEFAVCEDGKWFYDYRQLKNGM |
| | | | PKDPNGKKLSADEQIAYLEHLITKYPIDSIEDGLDEND |
| | | | WENWVKLTSAIGDRCQLVGDDLFVTNVKFLEKGIKMGA |
| | | | ANSILIKVNQIGSLTETLEAIEMAHRHGYTTVTSHRSG |
| | | | ETEDTTIADIAVATNSGQIKTGSMSRTDRMAKYNQLIR |
| | | | IEEELGACAKYGYAKLK |
| 61 | Outer_membrane_ | Q8G0Y6 | MKKLFTIAMLLGVTLGIHAQEVYSLQKCRELALQNNRQ |
| | efflux_protein_ | | LKVSRMTVDVAENTRKAAKTKYLPRVDALAGYQHFSRE |
| | Bepc | | ISLLSDDQKNAFSNLGTNTFGQLGGQIGQNLTSLAQQG |
| | | | ILSPQMAQQLGQLFSNVATPLTQVGNNIGQSINDAFRS |
| | | | NTKNVYAGGIVVNQPIYMGGAIKAANDMAAIGEQVAQN |
| | | | NISLKRQLVLYGVDNAYWLAISLKKKEALAIRYRDLAQ |
| | | | KLNEDVKKMIREGVATRADGLKVEVAVNTADMQIARIQ |
| | | | SGVSLAKMALCELCGLELNGDIPLSDEGDADLPPTPST |
| | | | QFDNYTVSSSDTTGLNEARPELRLLQNAVDLSIQNTKL |
| | | | IRSLYMPHVLLTAGYSVSNPNLFNGFQKRFTDLWNIGI |
| | | | TVQVPVWNWGENKYKVRASKTATTIAQLEMDDVRKKID |
| | | | LEIEQNRLRLKDANKQLATSQKNMAAAEENLRCANVGF |
| | | | KEGVMTVTEVMAAQTAWQTSRMAIIDAEISVKLAQTGL |
| | | | QKALGGL |
| 62 | Phosphoethanol- | Q7CPC0 | MKRTFVTKMVKPIEENSLFFMFMLLVGAFTNVSHRNVF |
| | amine_transferase_ | | GYIELIADVYIICFLLSLCQRTIRQGLVIMLSSVIYVV |
| | CptA | | AIIDTCCKTLFDTPITPTMLLLAQETTGREATEFFLQY |
| | | | LNLKLFFSAADIILFLAFCHIVMAVKKMKFSTSYLKQP |
| | | | FVAFVLMFTIFVGMALSIYDKVQLYTVKNLSGLEVAVT |
| | | | NGFAHLYHPVERIVYGLYSNHLIAKQVDGVIMANQQIK |
| | | | VDSCSFTSPTIVLVIGESANRHHSQLYGYPLPTTPYQL |
| | | | AMKNGKDSLAVFTNVVSPWNLTSKVFKQIFSLQSVDEK |
| | | | GDWSKYVLFPAVFKKAGYHVSFLSNQFPYGINYTPDWT |
| | | | NNLVGGFFLNHPQLNKQMFDYRNVTIHNYDEDLLNDYK |
| | | | EIISYKKPQLIIFHLLGQHFQYSLRCKSNMKKFGIKDY |
| | | | KRMDLTDKEKQTIADYDNATLYNDFVLNKIVEQFRNKD |
| | | | AIIVYLSDHGEDCYGKDVNMAGRLTEVEQINLKKYHEE |
| | | | FEIPFWIWCSPIYKQRHRKIFTETLMARNNKFMTDDLP |
| | | | HLLLYLAGIKTKDYCEERNVISPSFNNNRRRLVLKTID |
| | | | YDKALYQ |
| 63 | Dipeptide_and_ | P36837 | MFKNHPKGLLQAAFSNMGERFGYYIMNAVLALFLCSKF |
| | tripeptide_ | | GLSDETSGLIASLFLAAIYVMSLVGGVIADRTQNYQRT |
| | permease_B | | IESGLVVMALGYVALSIPVLATPENNSYLLAFTIFALV |
| | | | LIAVGNGLFKGNLQAIVGQMYDDFETEAAKVSPERLKW |
| | | | AQGQRDAGFQIFYVFINLGALAAPFIAPVLRSWWLGRN |
| | | | GLTYDAALPQLCHKYINGTIGDNLGNLQELATKVGGNS |
| | | | ADLASFCPHYLDVFNTGVHYSFIASVVTMLISLIIFMS |
| | | | SKKLFPMPGKKEQIVNVEYTDEEKASMAKEIKQRMYAL |
| | | | FAVLGISVFFWFSFHQNGQSLSFFARDFVNTDSVAPEI |
| | | | WQAVNPFFVISLTPLIMWVFAYFTKKGKPISTPRKIAY |
| | | | GMGIAGFAYLFLMGFSLVHNYPSAEQFTSLEPAVRATM |
| | | | KAGPMILILTYFFLTVAELFISPLGLSFVSKVAPKNLQ |
| | | | GLCQGLWLGATAVGNGFLWIGPLMYNKWSIWTCWLVFA |
| | | | IVCFISMVVMFGMVKWLERVTKS |
| 64 | C4- | Q9I4F5 | MQKKIKIGLLPRVIIAILLGLFLGYYLPDPAVRVFLTF |
| | dicarboxylate_ | | NSIFSQFLGFMIPLIIIGLVTPAIAGIGKGAGKLLLAT |
| | transport_ | | VAIAYVDTIVAGGLSYGTGTWLFPSMIASTGGAIPHID |
| | protein_2 | | KATELTPYFTINIPAMVDVMSSLVFSFIAGLGIAYGGL |
| | | | RTMENLFNEFKTVIEKVIEKAIIPLLPLYIFGVFLSMT |
| | | | HNGQARQVLLVFSQIIIVILVLHVLILIYEFCIAGAIV |
| | | | KHNPFRLLWNMLPAYLTALGTSSSAATIPVTLKQTVKN |
| | | | GVSEEVAGFVVPLCATIHLSGSAMKITACALTICMLTD |
| | | | LPHDPGLFIYFILMLAIIMVAAPGVPGGAIMAALAPLS |
| | | | SILGFNEEAQALMIALYIAMDSFGTACNVTGDGAIALA |
| | | | VNKFFGKKKETSILS |
| 65 | Inner_membrane_ | P76090 | MISVYSIKPQFQRVLTPILELLHRAKVTANQITLWACV |
| | protein_YnbA | | LSLVIGILFWFAGDVGTWLYLCLPVGLLIRMALNALDG |
| | | | MMARRYNQITRKGELLNEVGDVVSDTIIYFPLLKYHPE |
| | | | SLYFIVAFIALSIINEYAGVMGKVLSAERRYDGPMGKS |
| | | | DRAFVLGLYGVVCLFGINLSGYSVYIFGVIDLLLVLST |
| | | | WIRIKKTLKVTRNSQTPE |
| 66 | 2′,3′-cyclic- | P08331 | MKLSTILLSIMLGLSSSTMAQQKDVTIKLIETTDVHGS |
| | nucleotide | | FFPYDFITRKPKSGSMARVYTLVEELRKKDGKDNVYLL |
| | | | DNGDILQGQPISYYYNYVAPEKTNIAASVLNYMGYDVA |
| | | | TVGNHDIETGHKVYDKWFKELKFPILGANIIDTKTNKP |
| | | | YILPYYTIKKKNGIKVCVIGMLTPAIPNWLKESIWSGL |
| | | | RFEEMVSCAKRTMAEVKTQEKPDVIVGLFHSGWDGGIK |
| | | | TPEYDEDASKKVAKEVPGFDIVFFGHDHTPHSSIEKNI |
| | | | VGKDVICLDPANNAQRVAIATLTLRPKTVKGKRQYTVT |
| | | | KATGELVDVKELKADDAFIQHFQPEIDAVKAWSDQVIG |
| | | | RFENTIYSKDSYFGNSAFNDLILNLELEITKADIAFNA |
| | | | PLLFNASIKAGPITVADMFNLYKYENNLCTMRLTGKEI |
| | | | RKHLEMSYDLWCNTMKSPEDHLLLLSSTQNDAQRLGFK |
| | | | NFSFNFDSAAGIDYEVDVTKPDGQKVRILRMSNGEPFD |
| | | | ENKWYTVAVNSYRANGGGELLTKGAGIPRDSLKSRIIW |
| | | | ESPKDQRHYLMEEIKKAGVMNPQPNHNWKFIPETWTVP |
| | | | AAARDRKLLFGE |
| 67 | Fe(2+)_ | P33650 | MKLSELKTGETGVIVKVSGHGGFRKRIIEMGFIKGKTV |
| | transporter_FeoB | | EVLLNAPLQDPVKYKIMGYEVSLRHSEADQIEVLSDVK |
| | | | THSVGNEEEQEDNQLEMDSTTYDSTDKELTPEKQSDAV |
| | | | RRKNHTINVALVGNPNCGKTSLFNFASGAHERVGNYSG |
| | | | VTVDAKVGRAEFDGYVFNLVDLPGTYSLSAYSPEELYV |
| | | | RKQLVDKTPDVVINVIDSSNLERNLYLTTQLIDMHIRM |
| | | | VCALNMFDETEQRGDHIDAQKLSELFGVPMIPTVFTNG |
| | | | RGVKELFRQIIAVYEGKEDESLQFRHIHINHGHEIENG |
| | | | IKEMQEHLKKYPELCHRYSTRYLAIKLLEHDKDVEQLV |
| | | | SPLGDSIEIFNHRDTAAARVKEETGNDSETAIMDAKYG |
| | | | FINGALKEANFSTGDKKDTYQTTHVIDHVLTNKYFGFP |
| | | | IFFLVLLVMFTATFVIGQYPMDWIEAGVGWLGEFISKN |
| | | | MPAGPVKDMIVDGIIGGVGAVIVFLPQILILYFFISYM |
| | | | EDCGYMSRAAFIMDRLMHKMGLHGKSFIPLIMGFGCNV |
| | | | PAVMATRTIESRRSRLITMLILPLMSCSARLPIYVMIT |
| | | | GSFFALKYRSLAMLSLYIIGVLMAVAMSRLFSAFVVKG |
| | | | EDTPFVMELPPYRFPTWKAIGRHTWEKGKQYLKKMGGI |
| | | | ILVASIIVWALGYFPLPDDPNMDNQARQEQSYIGRIGK |
| | | | AVEPVFRPQGFNWKLDVGLLSGMGAKEIVASTMGVLYS |
| | | | NDGSFSDDNGYSSETGKYSKLHNLITKDVATMHHISYE |
| | | | EAEPIATLTAFSFLLFVLLYFPCVATIAAIKGETGSWG |
| | | | WALFAAGYTTALAWIVSAVVFQVGMLFM |
| 68 | UDP-N- | P9WJM1 | MESFIIEGGHQLSGTIAPQGAKNEALEVICATLLTSEE |
| | acetylglucosamine | | VIIRNVPDILDVNNLIKLLQDIGVKVKKLAPNEFSFQA |
| | | | DEVNLDYLESSDFVKKCSSLRGSVLMIGPLLGRFGKAT |
| | | | IAKPGGDKIGRRRLDTHFLGFKNLGAHFGRVEDRDVYE |
| | | | IQADKLVGTYMLLDEASITGTANIIMAAVLAEGTTTIY |
| | | | NAACEPYIQQLCKMLNAMGAKISGIASNLITIEGVKEL |
| | | | HSADHRILPDMIEVGSFIGIAAMIGDGVRIKDVSVPNL |
| | | | GLILDTFHRLGVQIIVDNDDLIIPRQDHYVIDSFIDGT |
| | | | IMTISDAPWPGLTPDLISVLLVVATQAQGSVLFHQKMF |
| | | | ESRLFFVDKLIDMGAQIILCDPHRAVVVGHDNAKKLRA |
| | | | GRMSSPDIRAGIALLIAALTAQGTSRIDNIVQIDRGYE |
| | | | NIEGRLNALGAKIQRAEVC |
| 69 | Ribitol-5- | Q8RKI9 | MNIAVIFAGGSGLRMHTKSRPKQFLDLNGKPIIIYTLE |
| | phosphate_cytidyl- | | LFDNHPNIDAIVVACIESWIPFLEKQLRKFEINKVVKI |
| | yltransferase | | IPGGKSGQESIYKGLCAAEEYAQSKGVSNEETTVLIHD |
| | | | GVRPLITEETITDNIKKVEEVGSCITCIPATETLIVKQ |
| | | | ADDALEIPSRADSFIARAPQSFRLIDIITAHRRSLAEG |
| | | | KADFIDSCTMMSHYGYKLGTIIGPMENIKITTPTDFFV |
| | | | LRAMVKVHEDQQIFGL |

In some embodiments, the Prevotella bacteria are from a strain of Prevotella bacteria comprising one or more of the proteins listed in Table 1 and that is free or substantially free of one or more proteins listed in Table 2. In some embodiments, the Prevotella bacteria are from a strain of Prevotella bacteria that comprises all of the proteins listed in Table 1 and/or all of the genes encoding the proteins listed in Table 1 and that is free of all of the proteins listed in Table 2 and/or all of the genes encoding the proteins listed in Table 2.

### Stabilizer and Bacterial Compositions

In some aspects, provided herein is a stabilizer that stabilizes bacterial compositions comprises sucrose. In some embodiments, the stabilizer comprises about 100 g/kg, about 110 g/kg, about 120 g/kg, about 130 g/kg, about 140 g/kg, about 150 g/kg, about 160 g/kg, about 170 g/kg, about 180 g/kg, about 190 g/kg, about 200 g/kg, about 210 g/kg, about 220 g/kg, about 230 g/kg, about 240 g/kg, about 250 g/kg, about 260 g/kg, about 270 g/kg, about 280 g/kg, about 290 g/kg, or about 300 g/kg sucrose. In some embodiments, the stabilizer comprises at least 100 g/kg, at least 110 g/kg, at least 120 g/kg, at least 130 g/kg, at least 140 g/kg, at least 150 g/kg, at least 160 g/kg, at least 170 g/kg, at least 180 g/kg, at least 190 g/kg, at least 200 g/kg, at least 210 g/kg, at least 220 g/kg, at least 230 g/kg, at least 240 g/kg, at least 250 g/kg, at least 260 g/kg, at least 270 g/kg, at least 280 g/kg, at least 290 g/kg, or at least 300 g/kg sucrose.

In some embodiments, the stabilizer comprises dextran 40k. In some embodiments, the stabilizer comprises about 100 g/kg, about 110 g/kg, about 120 g/kg, about 130 g/kg, about 140 g/kg, about 150 g/kg, about 160 g/kg, about 170 g/kg, about 180 g/kg, about 190 g/kg, about 200 g/kg, about 210 g/kg, about 220 g/kg, about 230 g/kg, about 240 g/kg, about 250 g/kg, about 260 g/kg, about 270 g/kg, about 280 g/kg, about 290 g/kg, or about 300 g/kg dextran 40k. In some embodiments, the stabilizer comprises at least 100 g/kg, at least 110 g/kg, at least 120 g/kg, at least 130 g/kg, at least 140 g/kg, at least 150 g/kg, at least 160 g/kg, at least 170 g/kg, at least 180 g/kg, at least 190 g/kg, at least 200 g/kg, at least 210 g/kg, at least 220 g/kg, at least 230 g/kg, at least 240 g/kg, at least 250 g/kg, at least 260 g/kg, at least 270 g/kg, at least 280 g/kg, at least 290 g/kg, or at least 300 g/kg dextran 40k.

In some embodiments, the stabilizer comprises cysteine HCl. In some embodiments, the stabilizer comprises about 1.0 g/kg, about 1.1 g/kg, about 1.2 g/kg, about 1.3 g/kg, about 1.4 g/kg, about 1.5 g/kg, about 1.6 g/kg, about 1.7 g/kg, about 1.8 g/kg, about 1.9 g/kg, about 2.0 g/kg, about 2.1 g/kg, about 2.2 g/kg, about 2.3 g/kg, about 2.4 g/kg, about 2.5 g/kg, about 2.6 g/kg, about 2.7 g/kg, about 2.8 g/kg, about 2.9 g/kg, about 3.0 g/kg, about 3.1 g/kg, about 3.2 g/kg, about 3.3 g/kg, about 3.4 g/kg, about 3.5 g/kg, about 3.6 g/kg, about 3.7 g/kg, about 3.8 g/kg, about 3.9 g/kg, about 4.0 g/kg, about 4.1 g/kg, about 4.2 g/kg, about 4.3 g/kg, about 4.4 g/kg, about 4.5 g/kg, about 4.6 g/kg, about 4.7 g/kg, about 4.8 g/kg, about 4.9 g/kg, or about 5.0 g/kg cysteine HCl. In some embodiments, the stabilizer comprises at least 1.0 g/kg, at least 1.1 g/kg, at least 1.2 g/kg, at least 1.3 g/kg, at least 1.4 g/kg, at least 1.5 g/kg, at least 1.6 g/kg, at least 1.7 g/kg, at least 1.8 g/kg, at least 1.9 g/kg, at least 2.0 g/kg, at least 2.1 g/kg, at least 2.2 g/kg, at least 2.3 g/kg, at least 2.4 g/kg, at least 2.5 g/kg, at least 2.6 g/kg, at least 2.7 g/kg, at least 2.8 g/kg, at least 2.9 g/kg, at least 3.0 g/kg, at least 3.1 g/kg, at least 3.2 g/kg, at least 3.3 g/kg, at least 3.4 g/kg, at least 3.5 g/kg, at least 3.6 g/kg, at least 3.7 g/kg, at least 3.8 g/kg, at least 3.9 g/kg, at least 4.0 g/kg, at least 4.1 g/kg, at least 4.2 g/kg, at least 4.3 g/kg, at least 4.4 g/kg, at least 4.5 g/kg, at least 4.6 g/kg, at least 4.7 g/kg, at least 4.8 g/kg, at least 4.9 g/kg, or at least 5.0 g/kg cysteine HCl.

In certain embodiments, the stabilizer is in liquid suspension. In some embodiments, the components of the stabilizer are dissolved in water to prepare the liquid suspension. In some such embodiments, the stabilizer comprises about 500 g/kg, about 510 g/kg, about 520 g/kg, about 530 g/kg, about 540 g/kg, about 550 g/kg, about 560 g/kg, about 570 g/kg, about 580 g/kg, about 590 g/kg, about 600 g/kg, about 610 g/kg, about 620 g/kg, about 630 g/kg, about 640 g/kg, about 650 g/kg, about 660 g/kg, about 670 g/kg, about 680 g/kg, about 690 g/kg, or about 700 g/kg water. In some such embodiments, the stabilizer comprises at least 500 g/kg, at least 510 g/kg, at least 520 g/kg, at least 530 g/kg, at least 540 g/kg, at least 550 g/kg, at least 560 g/kg, at least 570 g/kg, at least 580 g/kg, at least 590 g/kg, at least 600 g/kg, at least 610 g/kg, at least 620 g/kg, at least 630 g/kg, at least 640 g/kg, at least 650 g/kg, at least 660 g/kg, at least 670 g/kg, at least 680 g/kg, at least 690 g/kg, or at least 700 g/kg water.

In some embodiments, the stabilizer comprises sucrose, dextran 40k, cysteine HCl, and water. In some such embodiments, the stabilizer comprises 150 g/kg to 250 g/kg sucrose. In some embodiments, the stabilizer comprises 200 g/kg sucrose. In some embodiments, the stabilizer comprises 150 g/kg to 250 g/kg dextran 40k. In some embodiments, the stabilizer comprises 200 g/kg dextran 40 k. In some embodiments, the stabilizer comprises 2 g/kg to 6 g/kg cysteine HCl. In some embodiments, the stabilizer comprises 4 g/kg cysteine HCl. In some embodiments, the stabilizer comprises the stabilizer comprises 500 g/kg to 700 g/kg water. In some embodiments, the stabilizer comprises 596 g/kg water. In some embodiments, the stabilizer comprises 200 g/kg sucrose, 200 g/kg dextran 40k, 4 g/kg cysteine HCl, and 596 g/kg water.

In some aspects, provided herein are bacterial compositions comprising a stabilizer and bacteria, and methods of preparing same. In certain embodiments, the bacterial composition is prepared by combining bacteria with a certain percentage of the stabilizer in liquid suspension. In some embodiments, the percentage of the stabilizer solution combined with bacteria is about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In some embodiments, the percentage of the stabilizer solution combined with bacteria is at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, or at least 50%.

In certain aspects, the bacterial compositions provided herein comprise a stabilizer. In some embodiments, the bacterial composition comprises sucrose. In some embodiments, the concentration of sucrose in the bacterial composition is about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0%. In some embodiments, the concentration of sucrose in the bacterial composition is at least 0.1%, at least 0.2%, at least 0.3%, at least 0.4%, at least 0.5%, at least 0.6%, at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, at least 1.9%, at least 2.0%, at least 2.1%, at least 2.2%, at least 2.3%, at least 2.4%, at least 2.5%, at least 2.6%, at least 2.7%, at least 2.8%, at least 2.9%, or at least 3.0%.

In some embodiments, the bacterial composition comprises dextran 40k. In some embodiments, the concentration of dextran 40k in the bacterial composition is about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0%. In some embodiments, the concentration of dextran 40k in the bacterial composition is at least 0.1%, at least 0.2%, at least 0.3%, at least 0.4%, at least 0.5%, at least 0.6%, at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, at least 1.9%, at least 2.0%, at least 2.1%, at least 2.2%, at least 2.3%, at least 2.4%, at least 2.5%, at least 2.6%, at least 2.7%, at least 2.8%, at least 2.9%, or at least 3.0%.

In some embodiments, the bacterial composition comprises cysteine HCl. In some embodiments, the concentration of cysteine HCl in the bacterial composition is about 0.001%, about 0.005%, about 0.01%, about 0.011%, about 0.012%, about 0.013%, about 0.014%, about 0.015%, about 0.016%, about 0.017%, about 0.018%, about 0.019%, about 0.02%, about 0.021%, about 0.022%, about 0.023%, about 0.024%, about 0.025%, about 0.026%, about 0.027%, about 0.028%, about 0.029%, about 0.03%, about 0.031%, about 0.032%, about 0.033%, about 0.034%, about 0.035%, about 0.036%, about 0.037%, about 0.038%, about 0.039%, about 0.04%, about 0.041%, about 0.042%, about 0.042%, about 0.043%, about 0.044%, about 0.045%, about 0.046%, about 0.047%, about 0.048%, about 0.049%, or about 0.05%. In some embodiments, the concentration of cysteine HCl in the bacterial composition is at least 0.001%, at least 0.005%, at least 0.01%, at least 0.011%, at least 0.012%, at least 0.013%, at least 0.014%, at least 0.015%, at least 0.016%, at least 0.017%, at least 0.018%, at least 0.019%, at least 0.02%, at least 0.021%, at least 0.022%, at least 0.023%, at least 0.024%, at least 0.025%, at least 0.026%, at least 0.027%, at least 0.028%, at least 0.029%, at least 0.03%, at least 0.031%, at least 0.032%, at least 0.033%, at least 0.034%, at least 0.035%, at least 0.036%, at least 0.037%, at least 0.038%, at least 0.039%, at least 0.04%, at least 0.041%, at least 0.042%, at least 0.042%, at least 0.043%, at least 0.044%, at least 0.045%, at least 0.046%, at least 0.047%, at least 0.048%, at least 0.049%, or at least 0.05%.

In some embodiments, the bacterial composition comprises sucrose, dextran 40k, and cysteine HCl. In some such embodiments, the bacterial composition comprises 1% to 2% sucrose. In some embodiments, the bacterial composition comprises 1.5% sucrose. In some embodiments, the bacterial composition comprises 1% to 2% dextran 40k. In some embodiments, the bacterial composition comprises 1.5% dextran 40k. In some embodiments, the bacterial composition comprises 0.01% to 0.05% cysteine HCl. In some embodiments, the bacterial composition comprises 0.03% cysteine HCl. In some embodiments, the bacterial composition comprises 1.5% sucrose, 1.5% dextran 40k, and 0.03% cysteine HCl.

In certain aspects, the bacterial composition comprises bacteria. In some embodiments, the bacteria are anaerobic bacteria. In some embodiments, the anaerobic bacteria are Prevotella histicola. In some such embodiments, the anaerobic bacteria are Prevotella histocola Strain B 50329.

In some embodiments, the bacterial composition is lyophilized to form a powder.

### EXAMPLES

### Example 1: Exemplary Manufacturing Process of Prevotella histicola and Lyophilized Powder of Prevotella histicola and Stabilizer

Exemplary manufacturing processes of Prevotella histicola are shown in FIG. 1 and FIG. 2. In this exemplary method, the anaerobic bacteria are grown in growth media comprising the components listed in Table 3. The media is filter sterilized prior to use.

TABLE 3
Growth Media

| Component | g/L |
|---|---|
| Yeast Extract 19512 | 10 |
| Soy Peptone A2SC 19649 | 12.5 |
| Soy Peptone E110 19885 | 12.5 |
| Dipotassium Phosphate K2HPO4 | 1.59 |
| Monopotassium phosphate | 0.91 |
| L-Cysteine-HCl | 0.5 |
| Ammonium chloride | 0.5 |
| Glucidex 21 D (Maltodextrin) | 25 |
| Glucose | 10 |
| Hemoglobin | 0.02 |

Briefly, a 1 L bottle is inoculated with a 1 mL of a cell bank sample that had been stored at −80° C. This inoculated culture is incubated in an anaerobic chamber at 37° C., pH=6.5 due to sensitivity of this strain to aerobic conditions. When the bottle reaches log growth phase (after approximately 14 to 16 hours of growth), the culture is used to inoculate a 20 L bioreactor at 5% v/v. During log growth phase (after approximately 10 to 12 hours of growth), the culture is used to inoculate a 3500 L bioreactor at 0.5% v/v.

Fermentation culture is continuously mixed with addition of a mixed gas at 0.02 VVM with a composition of 25% CO₂ and 75% N₂. pH is maintained at 6.5 with ammonium hydroxide and temperature controlled at 37° C. Harvest time is based on when stationary phase is reached (after approximately 12 to 14 hours of growth).

Alternatively, the fermentation culture is continuously mixed with the addition of 100% CO₂ gas at 0.002 VVM. pH is maintained at 6.5 with ammonium hydroxide and temperature controlled at 37° C. Harvest time is based on when stationary phase is reached (after approximately 12 to 14 hours of growth).

Once fermentation is complete, the culture is cooled to 10° C., centrifuged, and the resulting cell paste is collected.

A stabilizer is prepared by combining and mixing the components described in Table 4. In order to prepare a lyophilized powder of Prevotella histicola and the stabilizer, 10% stabilizer is added to the cell paste and mixed thoroughly (Stabilizer Concentration (in slurry): 1.5% Sucrose, 1.5% Dextran, 0.03% Cysteine). The cell slurry is lyophilized.

TABLE 4
Stabilizer Formulation

| Component | g/kg |
|---|---|
| Sucrose | 200 |
| Dextran 40k | 200 |
| Cysteine HCl | 4 |
| Water | 596 |

### Example 2: Effect of CO₂ Availability on Prevotella histicola Growth

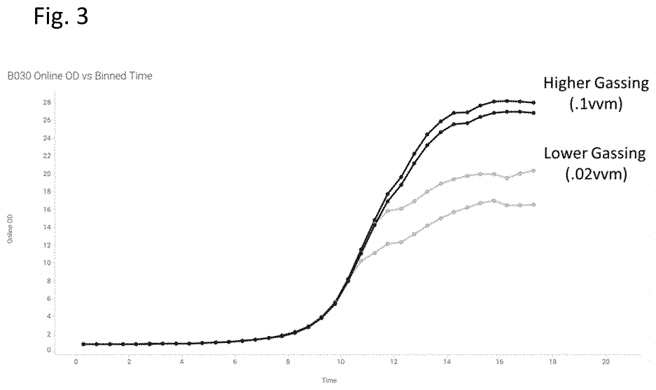

The effect of CO₂ availability on the growth of Prevotella histicola Strain B 50329 was tested. Prevotella histicola was cultured under anaerobic conditions with sparging of 95% N₂ and 5% CO₂ at a rate of either 0.1 volume of gas per volume of vessel per minute (vvm) or 0.02 vvm. As seen in FIG. 3, sparging an increased amount of the gas increased the growth potential of the Prevotella histicola.

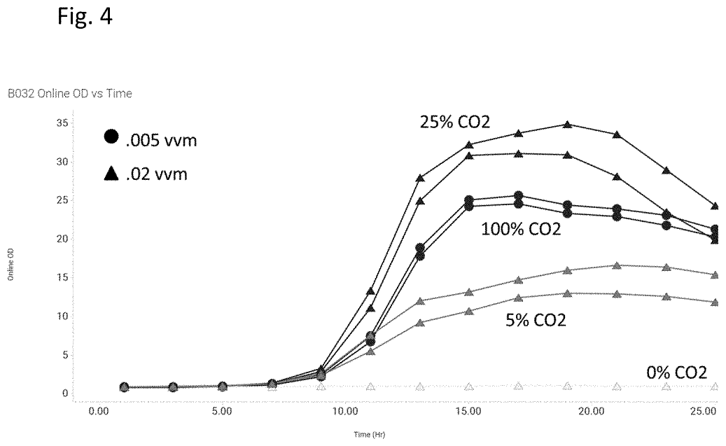

The Prevotella histicola strain was then cultured with sparging of pure N₂ (0% CO₂), 95% N₂ and 5% CO₂, or 75% N₂ and 25% CO₂ at a rate of 0.02 vvm. As can be seen in FIG. 4, the presence of CO₂ is necessary for initiation of Prevotella histicola growth. Sparging increasing concentrations of CO₂ increased the growth potential of the Prevotella histicola. Sparging 100% CO₂ at a lower rate (0.005 vvm) resulted in an intermediate growth potential for the Prevotella histicola.

At all scales, mass transfer of CO₂ is important and determined by a variety of factors. Here we show the impact of scale, agitation, gas concentration, and gas flow rate (Table 5).

TABLE 5
Prevotella histicola growth under various conditions

| | | CO₂ | Gas | Final | Final |
|---|---|---|---|---|---|
| Scale | Agitation | Conc. | flow rate | OD | TCC |
| 15L | 100 RPM | 5% CO₂ | 0.1vvm | 14.22 | 1.1 × 10¹⁰ |
| | | | | | cells/mL |
| 15L | 100 RPM | 5% CO₂ | 0.02vvm | 3 | NA |
| 15L | 100 RPM | 25% CO₂ | 0.02vvm | 11.7 | 7.16 × 10⁹ |
| | | | | | cells/mL |
| 36L | 50 RPM | 25% CO₂ | 0.02vvm | 10.99 | 8.47 × 10⁹ |
| | | | | | cells/mL |
| 50L | 100 RPM | 25% CO₂ | 0.02vvm | 30.1 | 3.77 × 10¹⁰ |
| | | | | | cells/mL |
| 50L | 60 RPM | 75% CO₂ | 0.007vvm | 32 | NA |
| 36L | 70 RPM | 25% CO₂ | 0.02vvm | 29 | 2.33 × 10¹⁰ |
| | | | | | cells/mL |
| 36L | 70 RPM | 25% CO₂ | 0.02vvm | 34.4 | 2.82 × 10¹⁰ |
| | | | | | cells/mL |
| 36L | 70 RPM | 25% CO₂ | 0.02vvm | 33.8 | 2.47 × 10¹⁰ |
| | | | | | cells/mL |

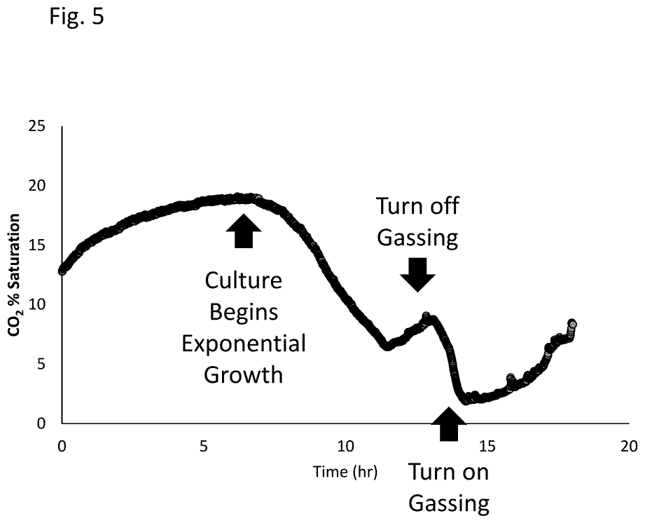

The Prevotella histicola was consuming CO₂ during growth. As seen in FIG. 5, when CO₂ was added to a freshly inoculated culture of Prevotella histicola, the CO₂ concentration increased and the concentration approached equilibrium. As the Prevotella histicola culture grew, the increase of CO₂ concentration slowed and then, as the culture entered logarithmic growth, the level of CO₂ declined. When the culture stopped logarithmic growth, this decline stopped as mass transfer offset consumption. When the sparging of the CO₂ was turned off, the concentration of CO₂ in the culture began to immediately to rapidly decline, indicating that that Prevotella histicola consumed CO₂. If no consumption were to occur, such as in sterile media, little to no change of CO₂ concentration would be observed in that time frame.

### Example 3: Maltodextrin in Combination with Glucose can Support Growth of Prevotella histicola Strain B 50329 Better than Glucose Alone as Sugar Source

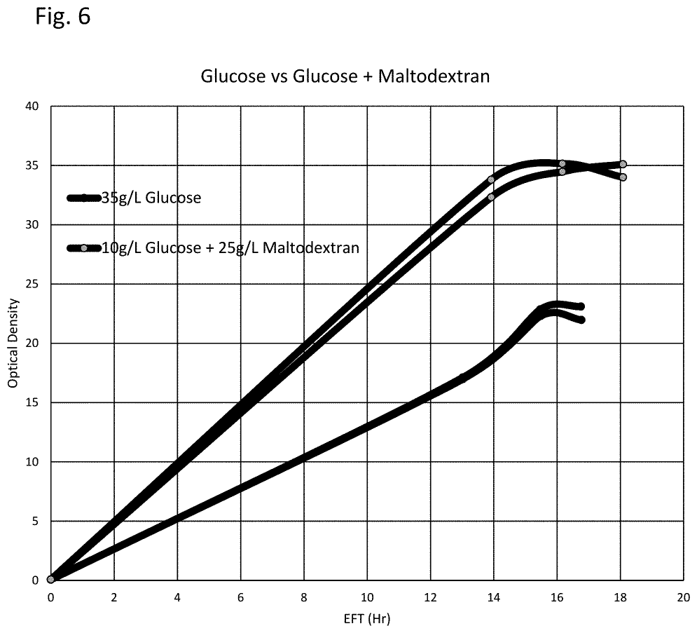

The results in FIG. 6 show that, at the same amount of total sugar, maltodextrin (25 g/L) in combination with glucose (10 g/L), compared to glucose (35 g/L) alone, led to increased process yield. Other than the sugars used, the culture conditions were identical. The result show that for equivalent masses of glucose and maltodextrin, Prevotella histicola Strain B 50329 grows better on maltodextrin plus glucose than on glucose alone. Because maltodextrin is just chains of glucose monomers, the results suggest that the cells may be benefiting in growth from some aspect of the chain structure.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of culturing anaerobic bacteria comprising culturing the anaerobic bacteria in a bioreactor under an anaerobic atmosphere comprising greater than 1% CO₂.

2. The method of claim 1, wherein the anaerobic atmosphere comprises at least 8% CO₂.

3. The method of claim 1 or claim 2, wherein the anaerobic atmosphere comprises at least 20% CO₂.

4. The method of claim 1, wherein the anaerobic atmosphere comprises from 8% to 40% CO₂.

5. The method of claim 1, wherein the anaerobic atmosphere comprises from 20% to 30% CO₂.

6. The method of claim 1, wherein the anaerobic atmosphere comprises about 25% CO₂.

7. The method of any one of claims 1-6, wherein the anaerobic atmosphere consists essentially of CO₂ and N₂.

8. The method of claim 1 wherein the anaerobic atmosphere comprises about 25% CO₂ and about 75% N₂.

9. A method of culturing anaerobic bacteria, the method comprising the steps of
a) purging a bioreactor with an anaerobic gas mixture comprising greater than 1% CO₂; and
b) culturing the anaerobic bacteria in the bioreactor purged in step a).

10. The method of claim 9, wherein the anaerobic gas mixture comprises at least 8% CO₂.

11. The method of claim 9 or claim 10, wherein the anaerobic gas mixture comprises at least 20% CO₂.

12. The method of claim 9, wherein the anaerobic gas mixture comprises from 8% to 40% CO₂.

13. The method of claim 9, wherein the anaerobic gas mixture comprises from 20% to 30% CO₂.

14. The method of claim 9, wherein the anaerobic gas mixture comprises about 25% CO₂.

15. The method of claim 9, wherein the anaerobic gas mixture comprises about 100% CO₂.

16. The method of any one of claims 9-15, wherein the anaerobic gas mixture consists essentially of CO₂ and N₂.

17. The method of claim 9, wherein the anaerobic gas mixture comprises about 25% CO₂ and about 75% N₂.

18. The method of any one of claims claim 9-17, wherein the method further comprises the step of inoculating a growth media with anaerobic bacteria, wherein the inoculation step precedes step b).

19. The method of claim 18, wherein the volume of anaerobic bacteria is about 0.1% v/v of the growth media.

20. The method of claim 19, wherein the growth media is about 1 L in volume.

21. The method of any one of claims 19-20, wherein the volume of anaerobic bacteria is about 1 mL.

22. The method of any one of claims 9-21, wherein the anaerobic bacteria is cultured for 10-24 hours.

23. The method of claim 22, wherein the anaerobic bacteria is cultured for 14 to 16 hours.

24. The method of any one of claims 22-23, wherein the method further comprises the step of inoculating about 5% v/v of the cultured bacteria in a growth media.

25. The method of claim 24, wherein the growth media is about 20 L in volume.

26. The method of any one of claims 24-25, wherein the anaerobic bacteria is cultured for 10-24 hours.

27. The method of claim 26, wherein the anaerobic bacteria is cultured for 12 to 14 hours.

28. The method of any one of claims 26-27, wherein the method further comprises the step of inoculating about 0.5% v/v of the cultured bacteria in a growth media.

29. The method of claim 28, wherein the growth media is about 3500 L in volume.

30. The method of any one of claims 28-29, wherein the anaerobic bacteria is cultured for 10-24 hours.

31. The method of claim 30, wherein the anaerobic bacteria is cultured for 12 to 14 hours.

32. The method of any one of claims 18-31, wherein the growth media comprises yeast extract, soy peptone A2SC 19649, Soy peptone E110 19885, dipotassium phosphate, monopotassium phosphate, L-cysteine-HCl, ammonium chloride, maltodextrin, glucose, and hemoglobin.

33. The method of claim 32, wherein the growth media comprises 5 g/L to 15 g/L yeast extract 19512.

34. The method of claim 32, wherein the growth media comprises 10 g/L yeast extract 19512.

35. The method of any one of claims 32-34, wherein the growth media comprises 10 g/L to 15 g/L soy peptone A2SC 19649

36. The method of claim 35, wherein the growth media comprises 12.5 g/L soy peptone A2SC 19649.

37. The method of any one of claims 32-36, wherein the growth media comprises 10 g/L to 15 g/L Soy peptone E110 19885.

38. The method of claim 37, wherein the growth media comprises 12.5 g/L Soy peptone E110 19885.

39. The method of any one of claims 32-38, wherein the growth media comprises 1 g/L to 2 g/L dipotassium phosphate.

40. The method of claim 39, wherein the growth media comprises 1.59 g/L Dipotassium phosphate.

41. The method of any one of claims 32-40, wherein the growth media comprises 0.5 g/L to 1.5 g/L monopotassium phosphate.

42. The method of claim 41, wherein the growth media comprises 0.91 g/L monopotassium phosphate.

43. The method of any one of claims 32-42, wherein the growth media comprises 0.1 g/L to 1.0 g/L L-cysteine-HCl.

44. The method of claim 43, wherein the growth media comprises 0.5 g/L L-cysteine-HCl.

45. The method of any one of claims 32-44, wherein the growth media comprises 0.1 g/L to 1.0 g/L ammonium chloride.

46. The method of claim 45, wherein the growth media comprises 0.5 g/L ammonium chloride.

47. The method of any one of claims 32-46, wherein the growth media comprises 20 g/L to 30 g/L maltodextrin.

48. The method of claim 47, wherein the growth media comprises 25 g/L maltodextrin.

49. The method of any one of claims 32-48, wherein the growth media comprises 5 g/L to 15 g/L glucose.

50. The method of claim 49, wherein the growth media comprises 10 g/L glucose.

51. The method of any one of claims 32-50, wherein the growth media comprises 0.01 g/L to 0.05 g/L hemoglobin.

52. The method of claim 51, wherein the growth media comprises 0.02 g/L hemoglobin.

53. The method of any one of claims 9-52, wherein the anaerobic bacteria is cultured at a temperature of 35° C. to 42° C.

54. The method of claim 53, wherein the anaerobic bacteria is cultured at a temperature of 37° C.

55. The method of any one of claims 9-54, wherein the anaerobic bacteria is cultured at a pH of 5.5 to 7.5.

56. The method of claim 55, wherein the anaerobic bacteria is cultured at a pH of 6.5.

57. The method of any one of claims 9-56, wherein culturing the anaerobic bacteria comprises agitating at a RPM of 50 to 300.

58. The method of claim 57, wherein the anaerobic bacteria is agitated at a RPM of 150.

59. The method of any one of claims 9-58, wherein the anaerobic gas mixture is continuously added during culturing.

60. The method of claim 59, wherein the anaerobic gas mixture is added at a rate of 0.002 vvm to 0.02 vvm.

61. The method of any one of claims 9-58, wherein CO₂ is continuously added during culturing.

62. The method of claim 61, wherein the CO₂ is added at a rate of 0.002 vvm to 0.1 vvm.

63. The method of claim 61, wherein the CO₂ is added at a rate of 0.007 vvm.

64. The method of claim 61, wherein the CO₂ is added at a rate of 0.1 vvm.

65. The method of any one of claims 9-64, wherein the method further comprising the step of harvesting the cultured bacteria when a stationary phase is reached.

66. The method of claim 65, further comprising the step of centrifuging the cultured bacteria after harvesting to produce a cell paste.

67. The method of claim 66, further comprising diluting the cell paste with a stabilizer solution to produce a cell slurry.

68. The method of claim 67, further comprising the step of lyophilizing the cell slurry to produce a powder.

69. The method of claim 68, further comprising irradiating the powder with gamma radiation.

70. The method of any one of claims 1-69, wherein the anaerobic bacteria are selected from bacteria of the genus Actinomyces, Bacteroides, Clostridium, Fusobacterium, Peptostreptococcus, Porphyromonas, Prevotella, Propionibacterium, or Veillonella.

71. The method of any one of claims 1-69, wherein the anaerobic bacteria are from a strain of Prevotella bacteria comprising one or more proteins listed in Table 1.

72. The method of any one of claims 1-69, wherein the anaerobic bacteria are from a strain of Prevotella substantially free of a protein listed in Table 2.

73. The method of any one of claims 1-69, wherein the anaerobic bacteria are from a strain of Prevotella bacteria comprising one or more of the proteins listed in Table 1 and is free or substantially free of a protein listed in Table 2.

74. The method of any one of claims 1-73, wherein the anaerobic bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

75. A bioreactor comprising anaerobic bacteria under an anaerobic atmosphere comprising at least about 1% CO₂.

76. The bioreactor of claim 75, wherein the anaerobic atmosphere comprises at least 8% CO₂.

77. The bioreactor of claim 75 or claim 76, wherein the anaerobic atmosphere comprises at least 20% CO₂.

78. The bioreactor of claim 75, wherein the anaerobic atmosphere comprises from 8% to 40% CO₂.

79. The bioreactor of claim 75, wherein the anaerobic atmosphere comprises from 20% to 30% CO₂.

80. The bioreactor of claim 75, wherein the anaerobic atmosphere comprises about 25% CO₂.

81. The bioreactor of any one of claims 75-80, wherein the anaerobic atmosphere consists essentially of CO₂ and N₂.

82. The bioreactor of claim 75, wherein the anaerobic atmosphere comprises about 25% CO₂ and about 75% N₂.

83. The bioreactor of any one of claims claim 75-82, wherein bioreactor is 1 L, 20 L, 3500 L 20,000 L, 50,000 L, 100,000 L, 200,000 L, 300,000 L, 400,000 L or 500,000 L.

84. The bioreactor of any one of claims 75-83, wherein the bioreactor further comprises a growth media.

85. The bioreactor of claim 84, wherein the growth media comprises yeast extract, soy peptone A2SC 19649, Soy peptone E110 19885, dipotassium phosphate, monopotassium phosphate, L-cysteine-HCl, ammonium chloride, glucidex 21 D, glucose, and hemoglobin.

86. The bioreactor of claim 85, wherein the growth media comprises 5 g/L to 15 g/L yeast extract 19512.

87. The bioreactor of claim 85 or claim 86, wherein the growth media comprises 10 g/L yeast extract 19512.

88. The bioreactor of any one of claims 85-87, wherein the growth media comprises 10 g/L to 15 g/L soy peptone A2SC 19649

89. The bioreactor of claim 88, wherein the growth media comprises 12.5 g/L soy peptone A2SC 19649.

90. The bioreactor of any one of claims 85-89, wherein the growth media comprises 10 g/L to 15 g/L Soy peptone E110 19885.

91. The bioreactor of claim 90, wherein the growth media comprises 12.5 g/L Soy peptone E110 19885.

92. The bioreactor of any one of claims 85-91, wherein the growth media comprises 1 g/L to 2 g/L dipotassium phosphate.

93. The bioreactor of claim 92, wherein the growth media comprises 1.59 g/L dipotassium phosphate.

94. The bioreactor of any one of claims 85-93, wherein the growth media comprises 0.5 g/L to 1.5 g/L monopotassium phosphate.

95. The bioreactor of claim 94, wherein the growth media comprises 0.91 g/L monopotassium phosphate.

96. The bioreactor of any one of claims 85-95, wherein the growth media comprises 0.1 g/L to 1.0 g/L L-cysteine-HCl.

97. The bioreactor of claim 96, wherein the growth media comprises 0.5 g/L L-cysteine-HCl.

98. The bioreactor of any one of claims 85-97, wherein the growth media comprises 0.1 g/L to 1.0 g/L ammonium chloride.

99. The bioreactor of claim 98, wherein the growth media comprises 0.5 g/L ammonium chloride.

100. The bioreactor of any one of claims 85-99, wherein the growth media comprises 20 g/L to 30 g/L glucidex 21 D.

101. The bioreactor of claim 100, wherein the growth media comprises 25 g/L glucidex 21 D.

102. The bioreactor of any one of claims 85-101, wherein the growth media comprises 15 g/L to 15 g/L glucose.

103. The bioreactor of claim 102, wherein the growth media comprises 10 g/L glucose.

104. The bioreactor of any one of claims 85-103, wherein the growth media comprises 0.01 g/L to 0.05 g/L hemoglobin.

105. The bioreactor of claim 104, wherein the growth media comprises 0.02 g/L hemoglobin.

106. The bioreactor of any one of claims 75-105, wherein the bioreactor is at a temperature of 35° C. to 42° C.

107. The bioreactor of claim 106, wherein the bioreactor is at a temperature of 37° C.

108. The bioreactor of any one of claims 85-107, wherein the growth media is at a pH of 5.5 to 7.5.

109. The method of claim 108, wherein the growth media is at a pH of 6.5.

110. The bioreactor of any one of claims 75-109, wherein the anaerobic bacteria are selected from bacteria of the genus Actinomyces, Bacteroides, Clostridium, Fusobacterium, Peptostreptococcus, Porphyromonas, Prevotella, Propionibacterium, or Veillonella.

111. The bioreactor of any one of claims 75-109, wherein the anaerobic bacteria are from a strain of Prevotella bacteria comprising one or more proteins listed in Table 1.

112. The bioreactor of any one of claims 75-109, wherein the anaerobic bacteria are from a strain of Prevotella substantially free of a protein listed in Table 2.

113. The bioreactor of any one of claims 75-109, wherein the anaerobic bacteria are from a strain of Prevotella bacteria comprising one or more of the proteins listed in Table 1 and is free or substantially free of a protein listed in Table 2.

114. The bioreactor of any one of claims 75-109, wherein the anaerobic bacteria are Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, or Prevotella veroralis.

115. A method of culturing anaerobic bacteria comprising culturing the anaerobic bacteria in a bioreactor into which a gas mixture comprising greater than 1% CO₂ is added.

116. The method of claim 115, wherein the gas mixture comprises at least 8% CO₂.

117. The method of claim 115 or claim 116, wherein the gas mixture comprises at least 20% CO₂.

118. The method of claim 115, wherein the gas mixture comprises from 8% to 40% CO₂.

119. The method of claim 115, wherein the gas mixture comprises from 20% to 30% CO₂.

120. The method of claim 115, wherein the gas mixture comprises about 25% CO₂.

121. The method of any one of claims 115-120, wherein the gas mixture consists essentially of CO₂ and N₂.

122. The method of claim 115 wherein the gas mixture comprises about 25% CO₂ and about 75% N₂.
